(19) United States
(12) Patent Application Publication
(10) Pub. No.: US 2021/0118547 A1
(43) Pub. Date: Apr. 22, 2021

(54) SYSTEMS, DEVICES, AND METHODS FOR SELF-CONTAINED PERSONAL MONITORING OF BEHAVIOR TO IMPROVE MENTAL HEALTH AND OTHER BEHAVIORALLY-RELATED HEALTH CONDITIONS

(71) Applicant: Singapore Ministry of Health Office for Healthcare Transformation, Singapore (SG)

(72) Inventors: Robert John Tasman MORRIS, Singapore (SG); Wijaya MARTANTO, Singapore (SG); Nikola VOUK, Singapore (SG); Xuancong WANG, Singapore (SG)

(21) Appl. No.: 16/905,833

(22) Filed: Jun. 18, 2020

Related U.S. Application Data
(60) Provisional application No. 62/924,132, filed on Oct. 21, 2019.

(51) Int. Cl. G16H 20/70; G16H 10/60; G16H 50/20; G16H 50/50; G16H 40/67; G16H 50/70; G16H 50/30; G16H 10/20; G16H 20/30; G16H 20/60; G16H 20/10; G06F 21/62; H04L 29/08; A61B 5/16; A61B 5/00; A61B 5/0205; A61B 5/11; A61B 5/0476; G09B 19/00

(52) U.S. Cl. CPC G16H 20/70; A61B 5/024; G16H 50/20; G16H 50/50; G16H 40/67; G16H 50/70; G16H 50/30; G16H 10/20; G16H 20/30; G16H 20/60; G16H 20/10; G06F 21/6245; H04L 67/141; A61B 5/165; A61B 5/7435; A61B 5/7264; A61B 5/0022; A61B 5/02055; A61B 5/4872; A61B 5/1112; A61B 5/1118; A61B 5/0476; A61B 5/167; A61B 5/4812; A61B 5/4815; G09B 19/00; G09B 19/0092; G16H 10/60

(57) ABSTRACT

A patient computing device and a set of behavioral and/or physiological parameter monitoring elements are configured for automatically: monitoring patient behavioral and/or physiological parameters over time; processing patient behavioral and/or physiological parameter data relative to a patient behavioral and/or mental health baseline state; determining whether a patient anomaly condition exists; and in response to the existence of a patient anomaly condition, automatically: determining a severity level corresponding to the anomaly condition; initiating execution of a behavioral therapy automaton; initiating execution of an automated patient dialog process that operates only on the patient computing device, without transfer of patient data and/or patient-identifying data external to the patient computing device; and/or selectively initiating data communication with an external electronic device or computing device or system corresponding to a care provider or care provider team associated with the patient in accordance with a set of patient pre-approved data communication permissions.

### CROSS-REFERENCE TO RELATED APPLICATION

This application claims priority to U.S. Provisional Patent Application No. 62/924,132, filed 21 Oct. 2019, which is incorporated by reference herein in its entirety.

### TECHNICAL FIELD

Aspects of the present disclosure relate to systems, devices, and methods for monitoring and processing the values of variables corresponding to individuals' behaviour(s) and/or physiological states, and estimating or determining aspects or measures of their behaviour(s). The disclosure further relates to interventions and maintenances that are useful in mental health and other behaviourally-related health conditions.

### BACKGROUND

The monitoring and processing of digital signals corresponding to a patient's behaviors and/or physiologic states, where such signals are derived from devices such as the patient's personal smartphone and/or wristband device, has become known as Digital Phenotyping. In association with Digital Phenotyping, measurements of the values of particular types of behavior related and/or physiologic state related variables are taken during the normal course of the patient's daily activities, such as talking on the phone, messaging, excursions constituting mobility, sleeping, resting, etc . . . Variables captured can include heart rate, movement-related activity (e.g., captured via accelerometer measurements), use of the phone for speech or messaging, geolocation data, etc. . .

It is known that several of these variables can be associated or correlated with the patient's mental health and wellness, and changes in such variables relative to a baseline state can be associated with abnormal patient symptoms. For example, in psychoaffective disorders, certain of these variables can exhibit increased or decreased values (e.g., relative to a normal patient population), according to negative or positive symptom groupings.

It is also well known that providing the patient with certain simple reminders can be effective in intercepting a decline in the patient's mental state. These reminders can be as simple as messages sent to the patient's mobile phone reminding the patient to take their medication(s), and/or engage in certain behavioral therapies such as “guided imagery.”

For patients experiencing particular mental health conditions, it is highly important that the patients retain their information privacy in association with their use of behavioral and/or physiological variable monitoring devices, and patients should not feel as if they are undergoing or could be subjected to excessive personal information disclosure or an excessive or intrusive level of surveillance. Hence, patients need to be able to trust the information privacy and security provisions associated with their monitoring device(s). Moreover, it is important that patients can trust the clinical or scientific relevance and/or effectiveness of the reminders and reminder-associated actions or activities in which they engage.

A need exists for secure, information privacy preserving, highly trusted systems, apparatuses, devices, and techniques for patient behavioral and mental state monitoring.

### SUMMARY

In accordance with an aspect of the present disclosure, a patient computing device and a set of behavioral and/or physiological parameter monitoring elements are configured for automatically: monitoring patient behavioral and/or physiological parameters over time; processing patient behavioral and/or physiological parameter data relative to a patient behavioral and/or mental health baseline state; determining whether a patient anomaly condition exists; and in response to the existence of a patient anomaly condition, automatically: determining a severity level corresponding to the anomaly condition; initiating execution of a behavioral therapy automaton; initiating execution of an automated patient dialog process that operates only on the patient computing device, without transfer of patient data and/or patient-identifying data external to the patient computing device; and/or selectively initiating data communication with an external electronic device or computing device or system corresponding to a care provider or care provider team associated with the patient in accordance with a set of patient pre-approved data communication permissions.

In accordance with an aspect of the present disclosure, a computerized process or method for non-revealing monitoring and processing of behavioral and/or mental state related information corresponding to a patient includes: (1) providing a patient computing device controlled by the patient, the patient computing device including a processing unit having or implemented by way of integrated circuitry and coupled to each of a memory, a set of input/output devices configured for providing a user interface, and a data communication unit, wherein the memory stores program instruction sets executable by the processing unit including program instruction sets corresponding to each of: (a) a patient behavioral and/or mental health management application program executable by the processing unit, which when executed provides visual or graphical interfaces by which the patient can interact with the patient computing device for self-management of their behavioral and mental health, and selectively communicate with devices and systems external to the patient computing device; (b) a set of patient behavioral/mental health variable monitoring modules, which when executed perform numerical/statistical operations upon patient behavioral, physiological, and/or mental health data; (c) a set of patient behavioral and/or mental health anomaly detection modules, which when executed perform anomaly detection operations in accordance with one or more machine learning or artificial intelligence models by which patient behavioral and/or mental health anomaly conditions can be automatically detected or recognized; (d) a behavioral therapy automaton; and (e) a data communication manager configured for managing or controlling data communication between the patient computing device and external systems, external devices, and data communication networks; (2) providing a set of patient behavioral and/or physiological parameter monitoring elements, each of which is configured for generating data correlated with the patient's behavioral and/or physiological state at a particular time or during a particular time period, wherein the set of patient behavioral and/or physiological parameter monitoring elements includes one or more electronic or computing devices configured for data communication with the patient computing device, and/or patient computing device hardware and/or program instruction sets; (3) initiating execution of the patient behavioral and mental health management application program; (4) receiving patient input by way of the set of input/output devices and establishing a set of patient data communication permissions that indicates types of patient behavioral and/or mental state information locally resident on the patient computing device that the patient computing device is permitted to communicate to destinations external to the patient computing device; (5) automatically monitoring patient behavioral and/or physiological parameters over a first time period and generating corresponding first patient behavioral and/or physiological parameter data by way of the set of patient behavioral and/or physiological parameter monitoring elements; (6) automatically processing the first patient behavioral and/or physiological data to determine a patient behavioral and/or mental health baseline state correlated with the first patient behavioral and/or physiological parameter data; (7) automatically monitoring patient behavioral and/or physiological parameters over another time period and generating corresponding additional patient behavioral and/or physiological parameter data by way of the set of patient behavioral and/or physiological parameter monitoring elements; (8) automatically processing the additional patient behavioral and/or physiological parameter data relative to the patient behavioral and/or mental health baseline state and determining whether a patient anomaly condition exists; (9) in response to the existence of a patient anomaly condition, at least one of automatically: (a) estimating or determining a severity level corresponding to the anomaly condition; (b) initiating execution of the behavioral therapy automaton and performing an automated behavioral therapy process based on the patient anomaly condition; (c) initiating execution of an automated patient dialog process that operates only on the patient computing device, without transfer of patient data and/or patient-identifying data external to the patient computing device; and (d) selectively initiating data communication with an electronic device or computing device or system corresponding to a care provider or care provider team associated with the patient in accordance with the set of patient data communication permissions.

The process or method typically includes automatically: repeating performing (6) through (9); and updating the patient behavioral and/or mental health baseline state over time based on one or more sets of additional patient behavioral and/or physiological parameter data.

Determining whether a patient anomaly condition exists can include identifying at least one of: a single variable anomaly corresponding to one patient behavioral and/or physiological parameter, and a multiple variable anomaly corresponding to at least two different patient behavioral and/or physiological parameters.

The set of patient data communication permissions can pre-approve transfer of at least some types of anonymized patient data external to the patient computing device, and the process or method can further include: automatically generating anonymized patient behavioral and/or mental health data correlated with at least portions of one or more sets of additional patient behavioral and/or physiological parameter data; and communicating the anonymized patient behavioral and/or mental health data to a remote computer system or remote data store associated with a behavioral and/or mental health care provider or care provider team for the patient in accordance with the set of patient data communication permissions.

Monitoring patient behavioral and/or physiological parameters over the first time period and the second time period can include monitoring at least some of patient: heart and/or pulse rate; body temperature; breathing rate; body weight; body fat percentage; blood pressure; geolocation; movement or mobility frequency, speed, range, and/or range variability; in-bed or sleep related parameters; circadian rhythms; electroencephalography (EEG) signals; and social media, Internet browser, short message service (SMS) messaging activity, and e-mail usage.

More particularly, monitoring patient behavioral and/or physiological parameters over the first time period and the second time period can include monitoring data corresponding to each of patient: sleep duration, sleep quality, mobility, and sociability.

The process or method can further include: presenting the patient with one or more behavioral and/or mental health surveys and/or questionnaires and receiving patient survey and/or questionnaire input corresponding thereto by way of the set of input/output devices; automatically processing the patient survey and/or questionnaire input and determining a current patient behavioral and/or mental health profile; and based on the processed patient survey and/or questionnaire input, downloading one or more of the set of patient behavioral/mental health variable monitoring modules, the set of patient behavioral and/or mental health anomaly detection modules, the behavioral therapy automaton, program instruction sets corresponding to a behavioral and/or mental health prescription, and one or more electronic behavioral and/or mental health lessons from a set of remote computing systems associated with a behavioral and/or mental health care provider for the patient.

The set of patient behavioral and/or mental health anomaly detection modules can be seeded to establish what is normal, what is normal variability, and what is anomalous for a group of individuals.

The behavioral and/or mental health prescription can include: a set of program instructions and/or a script executable by the processing unit, which establishes a sequence of automated behavioral therapy activities and/or electronic lessons in which the patient is to engage, and corresponding schedules for the automated behavioral therapy activities and/or electronic lessons; and optionally data, images, and/or videos corresponding to an exercise, dietary, and/or medication protocol that the patient is to follow.

The process or method can further include: determining that a patient anomaly condition is a recurring patient anomaly condition exists, which the patient recurrently or repeatedly experiences over time; and after determining that a recurring patient anomaly condition exists, at least one of: (a) automatically estimating a next recurrence time period; and (b) presenting the patient with one or more additional behavioral and/or mental health surveys and/or questionnaires and receiving additional patient input corresponding thereto by way of the set of input/output devices; automatically processing the additional patient input; and automatically adjusting or updating the behavioral and/or mental health prescription based on the processed additional patient input.

In accordance with an aspect of the present disclosure, a system for non-revealing monitoring and processing of behavioral and/or mental state related information corresponding to a patient includes: a patient computing device controlled by the patient, the patient computing device incudes a processing unit having or implemented by way of integrated circuitry and coupled to each of a memory, a set of input/output devices configured for providing a user interface, and a data communication unit, wherein the memory stores behavioral and/or health program instruction sets executable by the processing unit including program instruction sets corresponding to each of: (a) a patient behavioral and/or mental health management application program executable by the processing unit, which when executed provides visual or graphical interfaces by which the patient can interact with the patient computing device for self-management of their behavioral and mental health, and selectively communicate with devices and systems external to the patient computing device; (b) a set of patient behavioral and/or mental health variable monitoring modules, which when executed perform numerical or statistical operations upon patient behavioral, physiological, and/or mental health data; (c) a set of patient behavioral and/or mental health anomaly detection modules, which when executed perform anomaly detection operations in accordance with one or more machine learning or artificial intelligence models by which patient behavioral and/or mental health anomaly conditions can be automatically detected or recognized; (d) a behavioral therapy automaton; and (e) a data communication manager configured for managing or controlling data communication between the patient computing device and external systems, external devices, and data communication networks; and a set of patient behavioral and/or physiological parameter monitoring elements, each of which is configured for generating data correlated with the patient's behavioral and/or physiological state at a particular time or during a particular time period, wherein the set of patient behavioral and/or physiological parameter monitoring elements includes one or more electronic or computing devices configured for data communication with the patient computing device, and/or patient computing device hardware and/or program instruction sets, wherein the patient behavioral and/or mental health program instruction sets, when executed, cause the patient computing device to: (1) initiate execution of the patient behavioral and mental health management application program; (2) receive patient input by way of the set of input/output devices and establish a set of patient data communication permissions that indicates types of patient behavioral and/or mental state information locally resident on the patient computing device that the patient computing device is permitted to communicate to destinations external to the patient computing device; (3) automatically monitor patient behavioral and/or physiological parameters over a first time period and generating corresponding first patient behavioral and/or physiological parameter data by way of communication with the set of patient behavioral and/or physiological parameter monitoring elements; (4) automatically process the first patient behavioral and/or physiological data to determine a patient behavioral and/or mental health baseline state correlated with the first patient behavioral and/or physiological parameter data; (5) automatically monitor patient behavioral and/or physiological parameters over another time period and generating corresponding additional patient behavioral and/or physiological parameter data by way of communication with the set of patient behavioral and/or physiological parameter monitoring elements; (6) automatically process the additional patient behavioral and/or physiological parameter data relative to the patient behavioral and/or mental health baseline state and determining whether a patient anomaly condition exists; (7) in response to the existence of a patient anomaly condition, at least one of automatically: (a) estimate or determining a severity level corresponding to the anomaly condition; (b) initiate execution of the behavioral therapy automaton and performing an automated behavioral therapy process based on the patient anomaly condition; (c) initiate execution of an automated patient dialog process that operates only on the patient computing device, without transfer of patient data and/or patient-identifying data external to the patient computing device; and (d) selectively initiate data communication with an electronic device or computing device or system corresponding to a care provider or care provider team associated with the patient in accordance with the set of patient data communication permissions.

The patient behavioral and/or mental health program instruction sets, when executed, can further cause the patient computing device to repeatedly perform (5) through (7), and update the patient behavioral and/or mental health baseline state over time based on one or more sets of additional patient behavioral and/or physiological parameter data.

The set of patient behavioral and/or health anomaly detection modules can be configured for identifying at least one of: a single variable anomaly corresponding to one patient behavioral and/or physiological parameter, and a multiple variable anomaly corresponding to at least two different patient behavioral and/or physiological parameters.

The data communication module can be configured to control communication of patient data and patient-identifying data to destinations external to the patient computing device in accordance with the set of patient data communication permissions.

The set of patient behavioral and/or physiological parameter monitoring modules can be configured for monitoring at least some of patient: heart and/or pulse rate; body temperature; breathing rate; body weight; body fat percentage; blood pressure; geolocation; movement or mobility frequency, speed, range, and/or range variability; in-bed or sleep related motion; circadian rhythms; electroencephalography (EEG) signals; and social media, Internet browser, short message service (SMS) messaging activity, and e-mail usage.

The set of patient behavioral and/or physiological parameter monitoring modules can be configured for monitoring each of patient sleep patterns, patient mobility, and patient social media usage.

### BRIEF DESCRIPTION OF THE DRAWINGS

FIG. 1 is a block diagram of a system for self-contained personal and personalized monitoring and management of patient behavioral and mental health in accordance with particular embodiments of the present disclosure

FIGS. 2A-2B are block diagrams showing aspects of a set of server-side behavioral/mental health management resources in accordance with an embodiment of the present disclosure.

FIG. 3A illustrates a smartphone patient computing device 200 providing a first set of patient monitoring elements 110, and a wearable apparatus or device 205 providing a second set of patient monitoring elements in accordance with an embodiment of the present disclosure.

FIG. 3B is a block diagram showing further aspects of the smartphone and the first set of patient monitoring elements carried thereby in accordance with an embodiment of the present disclosure.

FIG. 4 is a graph corresponding to a manner of determining whether sleep quality anomaly exists based on the aforementioned variables in accordance with an embodiment of the present disclosure.

FIG. 5 is a graph corresponding to a manner of determining whether a mobility anomaly exists in accordance with an embodiment of the present disclosure.

FIG. 6A shows aspects of a first or simple deep learning based overall anomaly detection system in accordance with an embodiment of the present disclosure.

FIG. 6B shows aspects of a second or more complex deep learning based overall anomaly detection system in accordance with an embodiment of the present disclosure

FIGS. 7A-7B are flow diagrams showing aspects of a process for self-contained personal behavioral/mental health management in accordance with an embodiment of the present disclosure.

### DETAILED DESCRIPTION

In this specification, unless the context stipulates or requires otherwise, any use of the word “comprise,” and variations thereof such as “comprises” or “comprising,” imply the inclusion of a stated element or operation or group of elements or operations, but not the exclusion of any other element or operation or group of elements or operations.

The reference in this specification to any prior publication (or information derived from it), or to any matter which is known, is not, and should not be taken as an acknowledgment or admission or any form of suggestion that prior publication (or information derived from it) or known matter forms part of the common general knowledge in the field of endeavor to which this specification relates.

As used herein, the term “set” corresponds to or is defined as a non-empty finite organization of elements that mathematically exhibits a cardinality of at least 1 (i.e., a set as defined herein can correspond to a unit, singlet, or single element set, or a multiple element set), in accordance with known mathematical definitions (for instance, in a manner corresponding to that described in An introduction to Mathematical Reasoning: Numbers, Sets, and Functions, “Chapter 11: Properties of Finite Sets” (e.g., as indicated on p. 140), by Peter J. Eccles, Cambridge University Press (1998)). Thus, a set includes at least one element. In general, an element of a set can include or be one or more portions of a system, an apparatus, a device, a structure, an object, a process, a physical parameter, or a value depending upon the type of set under consideration.

Herein, reference to one or more embodiments, e.g., as various embodiments, many embodiments, several embodiments, multiple embodiments, some embodiments, certain embodiments, particular embodiments, specific embodiments, or a number of embodiments, need not or does not mean or imply all embodiments.

The FIGs. included herewith show aspects of non-limiting representative embodiments in accordance with the present disclosure, and particular elements shown in the FIGs. may be representative in nature, in that they are not shown to scale or precisely to scale relative to each other, and/or can be implemented in different or multiple manners. The depiction of a given element or consideration or use of a particular element number in a particular FIG. or a reference thereto in corresponding descriptive material can encompass the same, an equivalent, an analogous, categorically analogous, or similar element or element number identified in another FIG. or descriptive material associated therewith. The presence of “/” in a FIG. or text herein is understood to mean “and/or” unless otherwise indicated. The recitation of a particular numerical value or value range herein is understood to include or be a recitation of an approximate numerical value or value range, for instance, within +/−20%, +/−15%, +/−10%, +/−5%, +/−2.5%, +/−2%, +/−1%, +/−0.5%, or +/−0%. The term “essentially all” can indicate a percentage greater than or equal to 90%, for instance, 92.5%, 95%, 97.5%, 99%, or 100%.

Herein, the term “hardware” can include integrated circuitry, and the term “software” can include one or more program instruction sets that can be stored on or in a computer-readable or electronically-readable medium, and which are executable by a data processing unit or processing unit (e.g., integrated circuitry configurable or configured for executing stored program instructions, such as a set of microprocessors or microcontrollers). The term software can encompass or include firmware, in a manner readily understood by individuals having ordinary skill in the art. While particular elements may be embodied as or primarily as hardware or software, such elements can alternatively be embodied as or primarily as software or hardware, respectively, or a combination thereof, depending upon the type of element under consideration and/or embodiment details, in a manner readily understood by individuals having ordinary skill in the relevant art. The term “processing unit” can include integrated circuitry configured for performing automated data processing operations or implementing an automated data processor, such as a microprocessor or microcontroller that can execute stored program instructions to perform specific types of functions or operations, such as transforming input information or data into output information or data, in a manner readily understood by individuals having ordinary skill in the relevant art. The term “memory” can include one or more forms of random access memory (RAM) and/or read-only memory (ROM), in which data and/or program instructions can reside, in a manner readily understood by individuals having ordinary skill in the relevant art.

Overview

Embodiments in accordance with the present disclosure are directed to automated systems, sub-systems, devices, and processes for self-contained personal and personalized monitoring and management of patient behavioral and mental health, by which a specific human individual, patient, or subject can securely and privately monitor and manage their own behavioral and mental health states, conditions, patterns, and/or trends, without unnecessary or unwanted communication or transfer or revelation of personal data beyond electronic and data processing/computing resources (e.g., corresponding to hardware and/or software or program instruction sets, which can include firmware), apparatuses, or devices that they primarily, solely, or exclusively operate, configure, and control (e.g., by way of ownership thereof).

Various embodiments in accordance with the present disclosure are based upon non-revealing monitoring and processing of a specific patient's parameter or variable values of relevance or interest, and self-contained checking for anomalies in the patient parameter or variable values and/or patterns/trends, or relationships therebetween, by way of a computing device corresponding to this specific patient, and which is configured for executing a patient behavioral and mental health management application or app. In association with the execution of the behavioral and mental health management app, embodiments in accordance with the present disclosure ensure that the patient's personal data privacy is maintained by performing purely local analyses of privacy-sensitive data, and avoiding or preventing the transmission of privacy-sensitive data outside of the patient computing device. The patient computing device can perform inferencing operations based on a hand-crafted/patient-specific or multi-patient-derived model by way of machine learning, where such a model is downloaded from a remote server, such that the patient computing device acts as a client with respect to this server. Thus, the client patient computing device receives a downloaded model that establishes or describes what is normal, what is normal variability, and what is anomalous (e.g., for a group of patients).

This model therefore starts or is seeded with a simple model and technique/methodology/process for determining what is normal variability for an individual patient or group of patients. However, a further process, including but not limited to fundamental techniques of statistical variability and process control, establishes manners by which embodiments in accordance with the present disclosure can learn the baseline condition(s) of the patient and therefore determine excursion from the baseline(s), thereby constituting a designated anomaly.

Such analyses can yet be further refined based on what is learned (by way of machine learning) to be typical for this particular patient, and what are the normal variabilities for this patient. For instance, while there may be normal population-wide averages and variabilities for sleep duration across a population under consideration, a particular patient may function satisfactorily over a relatively long period of time by being in the lowest 5^{th} percentile of sleep duration, and this would not be flagged as abnormal for the patient under consideration. However, if this patient is observed as sleeping less than half of this amount over consecutive days, such a situation or condition could be flagged as anomalous.

Similarly, in sociability analysis there is a wide natural variability in the use of social networking or messaging between normal patients. Hence, while one patient who makes an average of 100 social media out-interactions per week, with a standard deviation of 40, would not be flagged as abnormal if they send only 30 messages in a given week, another patient who rarely or never sent messages may be flagged as hyperactive if they sent 30 messages over a 1-week time period.

By performing population analyses, further contextualized by patient specific analyses, embodiments in accordance with the present disclosure can identify or assess single variable or multi-variable anomalies. By performing joint or multi-variable analyses, embodiments in accordance with the present disclosure can achieve further refinement and improved specificity and sensitivity with respect to detecting and appropriately responding to anomalous patient behavioral and/or mental states. For instance, a patient that ceases going out in a given week, but engages in a normal or increased level of sociability may be suffering from the flu, yet keeping in touch with colleagues, family, and friends. However, a patient who is not going out, and has ceased all messaging with their messaging contacts may be unwell and could benefit, for example, from self-managed home therapy, or in other cases (when pre-approved by the patient according to their previously established privacy rules/guidelines/restrictions) from a trigger message sent to a clinical/care team who can initiate or establish or adjust the timing of a check-in call (e.g., a recurring or periodic check-in call) with the patient. Notwithstanding, in accordance with embodiments of the present disclosure, the trigger message for the check-in call would not disclose or indicate anything about the data (e.g., variable values as well as the results of variable value processing and analysis) that gave rise to the trigger message. Note that the concept of “non-revealing” may include selective revelations (e.g., by way of selective data communication or transfer), but only when pre-approved or consented to by the patient. This consenting can take place in the form of a policy, or be related to specific or one-time external transmissions that are specifically consented to by the patient (e.g., by way of a set of patient information/data transfer rules or restrictions, or analogously, a set of patient data communication permissions). This process of consent or approval applies to any trigger messages referred to above, and is further described below.

Particular embodiments in accordance with the present disclosure can perform personalized and contextualized decision making or decisioning, which can give rise to one or more categorical types of actions, such as the following:
1. Internal Actions (e.g., where “internal” can be defined as occurring only on a patient computing device such as a smartphone): Anomaly Detection can trigger a local automaton such as a “bot,” for instance, a chatbot, and pass signature to the bot/chatbot indicating which avenues of encouragement, enquiry, or previously-downloaded behavioral therapy (e.g., Cognitive Behavioral Therapy (CBT)) that the chatbot should pursue. No data is sent.

2. Internal Interaction with Resolution: The Anomaly Detection can trigger a completely self-contained dialog with the patient (e.g., where “self-contained” can be defined as occurring only on a patient computing device such as the patient's smartphone, with no data transfer or patient-identifying data transfer external to the smartphone), which resolves or explains the current concern. For example, the patient might respond to a dialog query with “I am abroad on vacation,” or “I am down with a cold this week,” and the answer may be processed/analyzed and deemed or judged sufficient to explain or clear a noted anomaly. However, the kind of answer, which can be entered by the patient's choice of options or natural language, may not be judged to be sufficiently explanatory and could lead to other actions.

3. External Approved Positive Action: The joint Anomaly Detection algorithm can be activated and an “I'm OK” message could be passed to the server. This accomplishes the purpose of assuring the clinical/care team that the patient is still undergoing monitoring processes or operations, and that the patient is doing satisfactorily and has not uninstalled or disabled their behavioral and mental health management application(s).

4. External Approved Moderated Action: The joint Anomaly Detection algorithm can be activated, and with the specific consent of the patient, the “I would like to interact with my clinical/care team” can be sent to the server (which is then routed to clinical/care team staff). The transmission of this message can also be voluntarily initiated by the patient by way of their personal computing device.

5. External Pre-approved Automated Action: When pre-authorized by the patient in accordance with or during an initial consenting process (e.g., which defines a set of data communication rules/restrictions pertaining to the transfer of patient data to networks, systems, devices, or destinations external to or remote from a patient computing device such as a smartphone), and under certain anomalous conditions, a “Request for Intervention” message can be sent to the server, without the need for prior explicit approval by the patient.

6. Distributed Learning Contribution: With the explicit consent of the patient, and for research purposes, a patient's data can be contributed anonymously (e.g., patient data or patient-related data is anonymized, such that no patient-identifying information is present or can be derived from the anonymized patient or patient-related data) towards the population-wide model. This aids in the distributed supervised or unsupervised learning of behavioral and/or mental health signatures, where various types of interventions may or may not be indicated.

In view of the foregoing, a specific individual patient can benefit from population-wide diagnostic and personalized and contextualized treatment models in a completely private context or setting, with only prior- and explicitly-consented data flowing back to the server or clinical/care team.

At patient onboarding time, and possibly one or more subsequent times, the patient may visit or converse with the clinical/care team, at which time further clinical observations may be obtained, taken, or recorded. This can result in one or more behavioral therapy (e.g., CBT) prescriptions, programs/apps/bots, or libraries thereof, which are provided and/or written by the clinical/care team and stored on a server associated therewith, being downloaded to the patient computing device.

Particular aspects or functional/operational capabilities in accordance with several embodiments of the present disclosure that are relevant to self-contained personal behavioral and mental health monitoring, e.g., by way of the execution of the patient behavioral and mental health management app, include:
1. A completely localized or self-contained patient monitoring system/methodology.
2. A completely localized coaching/behavioral therapy (e.g., CBT) system/methodology.
3. Localized coaching/behavioral therapy being triggered by local observations, and primed for the right kind of focus/foci and navigation.
4. The ability of the patient to both benefit from population-wide models of typical variability, learn locally what is typical or atypical for themselves, and adapt at their own pace (e.g., slowly) to changes in context, interests, and/or behaviors. Such changes could result from seasonal changes, or patient health improvement, or environmental changes such as a new job, school or commuting pattern, or a change in social circle(s) or a temporary geographical move corresponding to a vacation or relocation.
5. A corresponding ability to automatically identify and/or flag abnormal or anomalous behaviors, which can be addressed or cleared in a self-contained, private manner.
6. The ability to trigger (e.g., automatically trigger) the execution of a behavioral therapy (e.g., CBT) automaton (e.g., chatbot), and configure it to interact according to specific patient symptoms, monitored patient variable values, and/or patient variable anomalies or anomaly conditions (e.g., single and/or multiple variable anomalies or anomaly conditions), for instance, in response to the detection of poor patient sleep.
7. The ability to contact (e.g., automatically contact) the clinical/care team or a server associated therewith, but only when explicitly authorized by the patient.
8. The ability to communicate anomaly detection models to the server or clinical/care team, where such models contain coefficients but no personally identifiable information such as patient name, patient physical location, and patient data communication network address.
9. The ability to download a behavioral therapy (e.g., CBT) “prescription” (e.g., an app or bot for a given behavioral and/or mental health condition or state, such as positive thinking or stress management) to the patient computing device, where such a prescription has been selected or generated in collaboration with the clinical/care team.

Aspects of Particular System Configurations

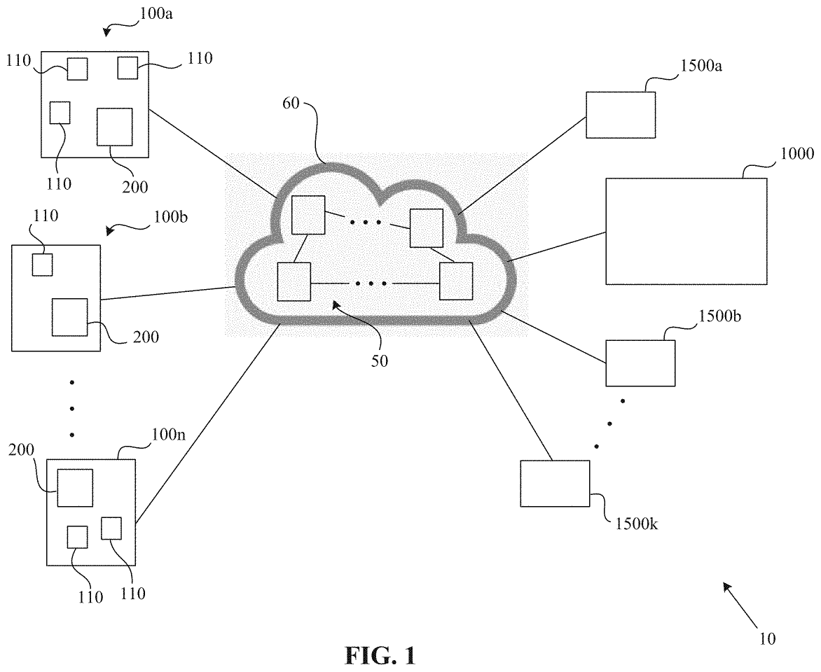

FIG. 1 is a block diagram of a system 10 for self-contained personal and personalized monitoring and management of patient behavioral and mental health in accordance with particular embodiments of the present disclosure. In an embodiment, the system 10 includes at least one set of automated patient-based or patient-side personal (and customizable or customized) behavioral/mental health management resources 100 (e.g., 100a . . . 100n), and at least one set of automated server-side or server-based behavioral/mental health management resources 1000, which are configured for data communication by way of one or more data communication networks 50, including or such as the Internet, a local area network (LAN), a wide area network (WAN), a satellite network, and/or a cellular network. The system 10 further includes at least one clinical/care team computer system 1500 (e.g., 1500a . . . 1500k) configured for data communication with the server-side or server-based behavioral/mental health management resources 1000, typically by way of the data communication network(s) 50. A given clinical/care team computer system 1500 can also be configured for data communication with one or more particular sets of patient-based or patient-side personal behavioral/mental health management resources 100, typically by way of the data communication network(s) 50, and which can possibly include intermediary data communication involving a particular set of server-side or server-based behavioral/mental health management resources 1000. One or more portions of the server-side or server-based behavioral/mental health management resources 1000 can be network-based, and can be hosted or reside in a public and/or private cloud 60, in a manner readily understood by individuals having ordinary skill in the relevant art.

A given set of patient-based or patient-side personal behavioral/mental health management resources 100 includes at least one patient computing device 200, which is configured for data communication (e.g., wireless and/or wire-based communication) with one or more patient behavioral and/or physiologic parameter or variable monitoring, measuring, or data capturing apparatuses, devices, elements, units, or modules 110, as further elaborated upon below.

Aspects of Server-Side Behavioral/Mental Health Management Resources

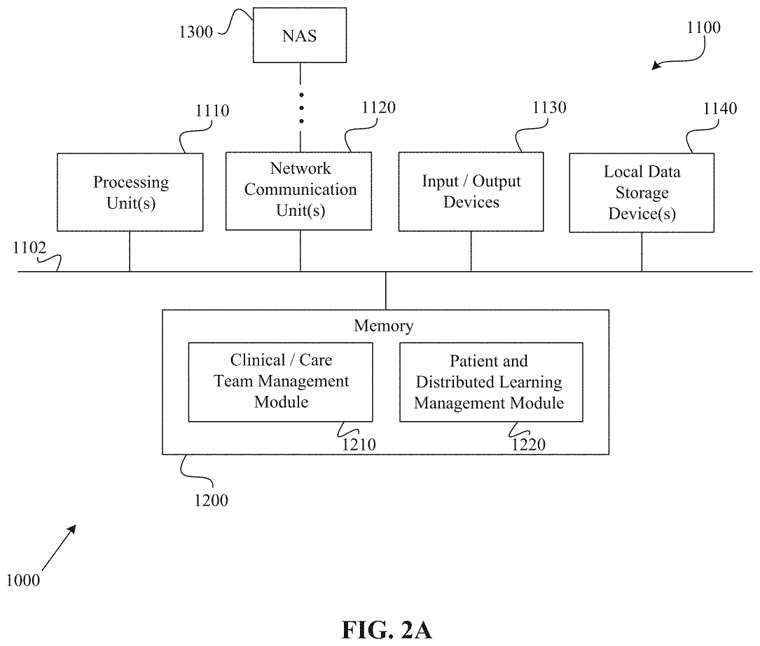

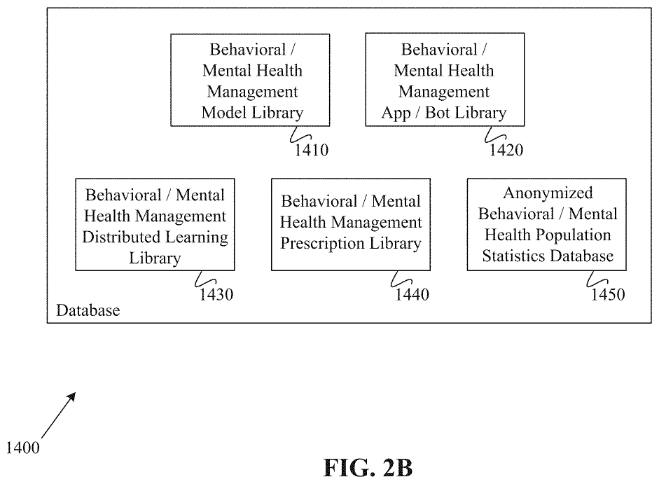

FIGS. 2A-2B are block diagrams showing aspects of a set of server-side behavioral/mental health management resources 1000 in accordance with an embodiment of the present disclosure. In an embodiment, a particular set of server-side behavioral/mental health management resources 1000 includes one or more computer systems such as a set of servers 1100, and at least one data storage system or unit 1300.

A given server 1100 can provide or include one or more processing units 1110 (e.g., microprocessors); at least one network communication unit 1120; a set of input/output devices 1130; a set of local data storage devices 1140 (e.g., disk drives); and at least one memory 1200 in which data and program instruction sets executable by the processing unit(s) 1110, including an operating system, can reside. A server 1100 under consideration is typically configured for data communication with a particular set of data storage units 1300, such as one or more network attached storage (NAS) systems or units 1300 with which the server 1100 can communicate by way of its network communication unit(s) 1120. Portions of one or more databases can reside in or across the memory 1200, the local data storage device(s) 1140, and/or the data storage unit(s) 1300, in a manner readily understood by individuals having ordinary skill in the relevant art. The memory 1200 and the data storage units 1300 can provide or form portions of electronically or computer readable media on and/or in which data and program instruction sets can be stored, in a manner readily understood by individuals having ordinary skill in the relevant art. Each of the foregoing elements can be configured for communication or coupled by way of a particular data transfer or communication pathways 1102, in a manner individuals having ordinary skill in the relevant art will readily comprehend.

The memory 1200 includes a clinical/care team management module 1210; and a patient management and distributed learning module 1220, each of which can include or be portions of an application program executable by the processing unit(s) 1110. The clinical/care team management module 1210 is configured for managing clinical/care team computer system communication and interaction with the server 1100 and the data storage unit(s) 1300 associated therewith; and similarly, the patient management and distributed learning module 1220 is configured for managing patient computing device communication and interaction with the server 1100 and the data storage unit(s) 1300 associated therewith.

FIG. 2B is a block diagram illustrating aspects of a behavioral/mental health database 1400 that can exist within portions of the memory 1200, the local data storage device(s) 1440, and/or the data storage unit(s) 1300 in accordance with an embodiment of the present disclosure. In an embodiment, the database 1400 includes a behavioral/mental health management model library 1410, which contains software-based numerical seeding models that are executable by a patient computing device 200 for modeling, estimating, or determining particular aspects of a patient's behavioral and mental health; a behavioral/mental health management app/bot library 1420, which includes software-based behavioral therapy (e.g., CBT) apps/bots (e.g., chatbots) executable by a patient computing device 200 for aiding the patient in self-managing their behavioral and mental health, or symptoms associated therewith; a behavioral/mental health distributed learning library 1430, which includes educational materials and lessons that can be communicated to patient computing devices 200 (e.g., in association with or in the form of apps executable thereby) to aid patient self-management of their behavioral and mental health, or symptoms associated therewith; a behavioral/mental health management prescription library 1440, which includes software-based behavioral/mental health prescriptions that are executable by a patient computing device 200 as part of patient management of their behavioral and mental health (e.g., and which can be associated or linked with or initiate the execution of specific behavioral/mental health apps/bots), and which are selectable, customizable or customized by, and/or defined or written by a clinician or clinical/care team member for their patients by way of their clinical/care team computer system 1500; and an anonymized patient behavioral/mental health population statistics database 1450, which includes statistical data based upon or derived from patient-anonymized behavioral and/or mental health parameter or variable values that are correlated with or clinically relevant with respect to patient behavioral and mental health.

Aspects of Patient-Side Behavioral/Mental Health Management Resources

With reference again to FIG. 1, each set of patient-based or patient-side personal behavioral/mental health management resources 100 includes hardware and/or software corresponding to a number of automated electronic and/or data processing/computing devices that are configurable or configured for monitoring and managing aspects of a specific patient's behavioral and/or mental health by way of performing or executing processes and operations (e.g., in accordance with execution of program instruction sets) in a manner that maintains patient data security, and enhances, greatly increases, or maximizes patient privacy (e.g., patient data collection, analysis, and communication privacy). More particularly, such automated electronic and/or data processing/computing devices correspond to and are under primary or direct control of (e.g., are owned by) a specific patient (e.g., only that patient). A given set of patient-based or patient-side personal behavioral/mental health management resources 100 is configured for receiving and/or acquiring input and/or data that is, is expected to be, or can be associated or correlated with one or more of the specific patient's current, recent, or long-term behavioral and/or mental health states; processing or analyzing such input and/or data; and generating or providing output, executing behavioral therapy (e.g., CBT) program instruction sets (e.g., which form portions of behavioral therapy application programs or apps, and which can include automata such as bots, for instance, chatbots), and providing user interfaces (e.g., visual or graphical user interfaces) that enable the specific patient to which this set of patient-based or patient-side personal behavioral/mental health management resources 100 corresponds to substantially or essentially entirely manage aspects of their behavioral/mental health on their own, e.g., independently, without unnecessarily or undesirably compromising their personal data security and privacy. For purpose of simplicity and clarity in the description that follows, a set of patient-based or patient-side personal behavioral/mental health management resources 100 can be referred to as a set of personal behavioral/mental health management resources 100.

As indicated above, a set of personal behavioral/mental health management resources 100 includes at least one patient behavior and/or physiologic variable monitoring, measuring, or data capturing apparatus, device, element, unit, or module 110 corresponding to and under primary or direct control of a specific patient (e.g., only that patient), and at least one patient computing apparatus, device, element, unit, or module 200 corresponding to and under primary or direct control of this specific patient. In the following description, a patient behavior and/or patient physiologic parameter monitoring, measuring, or data capturing apparatus, device, element, unit, or module 110 can simply be referred to as a patient monitoring element 110; and a patient computing apparatus, device, element, unit, or module 200 can simply be referred to as a patient computing device 200. Within a particular set of personal behavioral/mental health management resources 100, the patient monitoring element(s) 110 are configured for data communication with the patient computing device(s) 200.

A given patient monitoring element 110 can include hardware and/or software depending upon the type of patient monitoring element under consideration and/or embodiment details, in a manner readily understood by individuals having ordinary skill in the relevant art. A patient monitoring element 110 includes or is configured for monitoring, acquiring, sensing, measuring, estimating, deriving, or determining (hereafter monitoring for purpose of simplicity and clarity) and storing data (e.g., data values) corresponding to or indicative of one or more types of behavior related and/or mental/physiologic state related parameters or variables for the specific patient under consideration over time (e.g., on an ongoing, recurring, periodic, or generally continuous basis across multiple minutes, hours, days, weeks, months, and/or years). Depending upon embodiment details, a particular set of patient monitoring elements 110 can be configured for monitoring patient-specific parameters or variables such as patient: heart/pulse rate; body temperature; breathing rate; body weight; body fat percentage; blood pressure; geolocation; movement or mobility frequency, speed, range, and/or range variability (e.g., as indicated in association with or by a set of accelerometers and/or gyroscopes); sleep related variables such as motion during sleep and circadian rhythms; electroencephalography (EEG) signals (e.g., captured by way of a patient-worn EEG headset); social media, Internet browser, short message service (SMS) messaging activity, and/or e-mail usage (e.g., access or viewing times) and incoming/outbound data transfer measures or metrics associated therewith; and/or other patient-specific variables. A patient monitoring element 110 can include or be one or more portions of a mobile phone; a patient-wearable device such as a wrist-worn/wristband, arm-worn, or leg-worn type of device (e.g., generally similar, similar, or analogous to a Fitbit® (Fitbit Inc., San Francisco, Calif. USA) device or an Apple Watch® (Apple Inc., Cupertino, Calif. USA) device), or a foot-worn device (e.g., a pair of shoes having a set of sensors therein configured for monitoring certain patient-specific motion or movement-related variables); another type of a patient transportable/patient carriable device (e.g., a walking stick or cane having a set of sensors therein configured for monitoring certain patient-specific motion or movement-related variables); or another type of device such as a digital weight and body fat percentage scale, or a blood pressure cuff, located in the patient's home.

A particular patient computing device 200 corresponding to a given set of patient monitoring elements 110 includes hardware and/or software based data processing/computing resources configured for processing and analyzing patient-specific behavioral and/or mental health variables corresponding to data or data values obtained by way of the patient monitoring element(s) 110 to estimate or determine tendencies, patterns, or trends exhibited by and relationships between the patient-specific variables (e.g., corresponding to or represented over time as variable data values) with respect to one or more time intervals, periods, or scales, which can be or are expected to be correlated with the patient's behavioral and/or mental health state(s) over time. A patient computing device 200 is further configured for (a) providing feedback and/or automated behavioral therapy programs, scripts, or exercises to the patient based on the processing and analysis of such patient-specific variables; and in particular circumstances or in response to certain events (e.g., trigger events), (b) communicating with a set of server-side behavioral/mental health management resources 1000 and/or a computing device associated with a clinical/care team. A patient computing device 200 can typically include or be, for instance, a mobile phone/smartphone, a tablet computer, a laptop computer, a desktop computer.

In view of the foregoing, individuals having ordinary skill in the relevant art will understand that a patient computing device 200 can include or carry one or more patient monitoring elements 110; and a patient monitoring element 110 can include one or more data processing/computing resources. For instance, a patient computing device 200 such as a smartphone can be equipped with patient monitoring elements 110 such an accelerometer/gyroscope unit; a geolocation unit; one or more social media and/or e-mail apps; and/or additional or other patient monitoring elements 110. A patient monitoring element 110 carried by a wristband-type device can be configured for monitoring patient movement and/or sleep related variable values, and can include a processing unit (e.g., a microprocessor or microcontroller), a memory storing a control program or app executable by the processing unit to process and analyze such variable values with respect to particular time periods, and a data communication unit configured for wireless and/or wire-based data transfer.

In accordance with various embodiments of the present disclosure, the patient monitoring element(s) 110 and the patient computing device(s) 200 within a given set of personal behavioral/mental health management resources 100 perform processes and operations associated with or relevant to monitoring and/or managing a specific patient's behavioral and mental health without unnecessarily revealing or communicating, or revealing or communicating without the patient's explicit consent or permission in the absence of an emergency or likely emergency situation, each of:
(a) the data content or values of monitored patient-specific variables and the results of processing such variable values, and
(b) patient inputs and responses associated with the execution of behavioral therapy apps (e.g., CBT chatbots)

to destinations or devices external to the patient computing device(s) 200 and this set of patient monitoring elements 110.

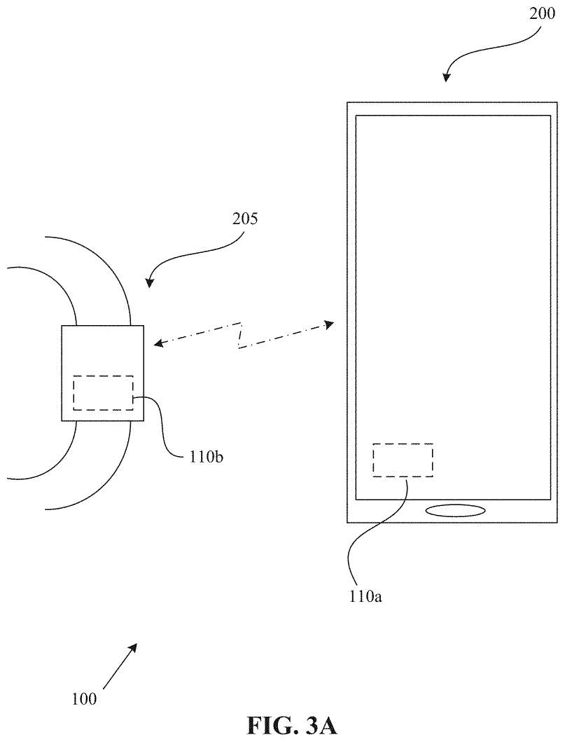

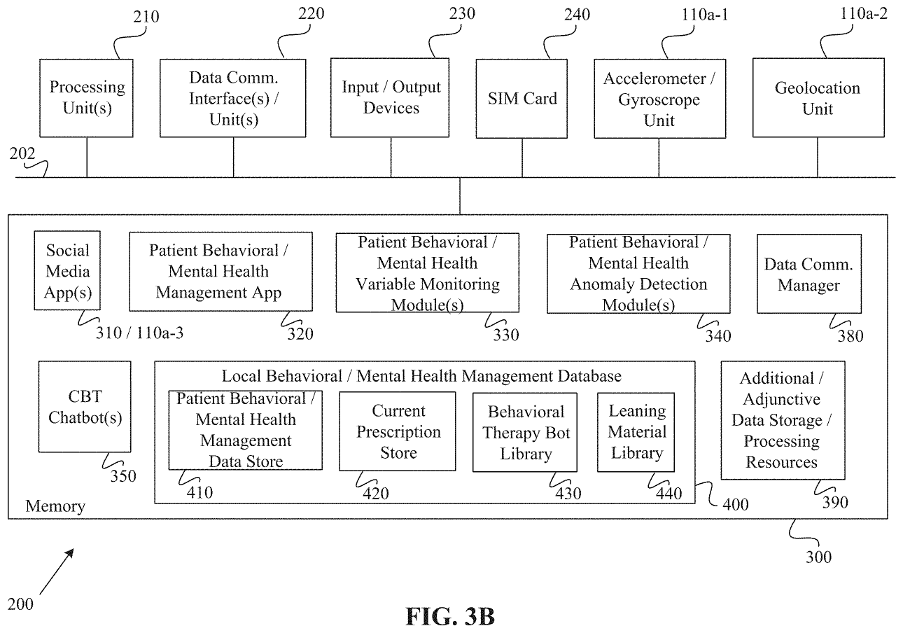

In view of the foregoing, FIG. 3A illustrates a non-limiting representative embodiment of a patient computing device 200 and patient monitoring elements 110 in accordance with the present disclosure, which is considered herein for purpose of simplicity and to aid understanding. In this representative embodiment, a patient computing device 200 includes or is a smartphone/mobile phone 200 (hereafter smartphone 200), which typically provides or carries a first set of patient monitoring elements 110a configured for monitoring at least (a) patient mobility and movement related variable values, and (b) patient sociability related variable values. A second set of patient monitoring devices 110b resides external to the smartphone/mobile phone 200, and is configured for monitoring at least patient sleep related variable values, possibly or typically in association with or based on patient movement related variable values. The second set of patient monitoring devices 110b can be carried by a patient-wearable apparatus or device 205, such as a wrist-worn, arm-worn, leg-worn, torso-worn, or head-worn device. Such a wearable apparatus or device 205 can include computing/data processing resources (e.g., a data processing unit such as a microprocessor or microcontroller; a memory; a set of input/output devices; and a data communication unit), and can thus be categorized or defined as a type of patient computing device 200, in a manner readily understood by individuals having ordinary skill in the art. The second set of patient monitoring devices 110b is configured for wireless (e.g., Wi-Fi and/or Bluetooth®) and/or wire-based (e.g., Universal Serial Bus (USB)) communication with the smartphone 200.

FIG. 3B is a block diagram showing further aspects of a patient computing device 200 such as a smartphone 200 as mentioned above, and the first set of patient monitoring elements 110a-1,2,3 carried thereby in accordance with such a representative embodiment of the present disclosure. The smartphone 200 includes a processing unit 210; a memory 300 in which data and program instructions executable by the processing unit 210, including an operating system, can reside; a set of data communication interfaces/units (e.g., a Wi-Fi unit and/or a Bluetooth® unit, and a USB communication interface) 220; a set of input/output devices 230, such as a touch-sensitive display screen, a microphone, a speaker, and user control elements associated therewith; at least one Subscriber Identity Module (SIM) card 240; an accelerometer/gyroscope unit 110a-1; and a geolocation unit (e.g., a global positioning satellite (GPS) or similar unit) 110a-2.

The memory 300 includes program instruction sets or program instruction modules that are executable by the processing unit 210, and which include at least one social media app 310, which also serves as a patient monitoring element 110a-3; a patient behavioral/mental health management app 320, which when executed provides visual or graphical interfaces by which the patient can interact with their smartphone 200 for self-management of their behavioral and mental health, and selectively communicate with a particular clinical/care provider computer system 1500; a set of patient behavioral/mental health variable monitoring modules 330, which can include program instruction sets which when executed perform numerical/statistical operations upon behavioral/mental health variable data (e.g., data values); a set of patient behavioral/mental health anomaly detection modules 340, which can include program instruction sets which when executed perform operations in accordance with one or more machine learning or artificial intelligence models by which patient behavioral/mental health anomalies can be automatically detected or recognized; possibly one or more behavioral therapy apps/bots (e.g., a CBT chatbot) 350; a data communication manager 380 configured for managing or controlling data communication between the smartphone and external or remote/non-local systems, devices, and the data communication networks 50, e.g., involving data communication between the smartphone 200 and non-local or remote server-side behavioral/mental health management resources 1000 and/or a clinical/care team computer system 1500; and a local behavioral/mental health management database 400 that includes a patient behavioral/mental health management data store 410 (e.g., for storing one or more types of data such as patient behavioral/mental health/physiological parameter data values); a current prescription store 420 in which one or more current patient prescriptions can reside; a behavioral therapy app/bot (e.g., CBT bot) library 430; and a learning material library 440.

The patient computing device/smartphone 200 can also include further hardware and/or software resources, for instance, a set of additional/adjunctive data storage and/or data processing resources 390, which in various embodiments includes (a) a data encryption/decryption module configured for generating encryption/decryption keys, and encrypting/decrypting (i) patient data, (ii) patient-identifying data, and (iii) possibly anonymized patient-related data; and (b) a secure encryption/decryption key store in which a set of patient-specific encryption/decryption keys (e.g., a set of private keys and corresponding public keys) corresponding to the patient reside. Such further hardware and/or software resources can also include a set of patient biometric data capture devices (e.g., a patient fingerprint capture device), such that patient biometric data can be used in an encryption key generation process or procedure, in a manner readily understood by individuals having ordinary skill in the relevant art. Based on the set of patient information/data transfer rules or restrictions, or analogously, the set of patient data communication permissions, the data communication manager 380 can communicate a set of decryption keys to a clinical/care team computer system 1000 such that one or more types of encrypted patient-related or patient-derived data can be decrypted in a manner that is explicitly pre-approved by the patient, as individuals having ordinary skill in the relevant art will also readily comprehend. In embodiments in which the patient computing device/smartphone 200 generates and stores encrypted patient-related or patient-derived data, the patient behavioral/mental health management app 320, the patient behavioral/mental health variable monitoring module(s) 330, and the patient behavioral/mental health anomaly detection module(s), and/or possibly the CBT chatbot(s) 350 can utilize the encryption/decryption module for encrypting and decrypting particular patient-related or patient-derived data in association with performing self-contained personal behavioral/mental health management processes in accordance with various embodiments of the present disclosure.

Each of the foregoing elements of the smartphone 200 can be configured for communication or coupled by way of a particular data transfer or communication pathways 1102, in a manner individuals having ordinary skill in the relevant art will readily comprehend. Individuals having ordinary skill in the relevant art will recognize that the smartphone 200 (e.g., by way of execution of the patient behavioral/mental health management app 310) can function as a client with respect to one or more servers 1100 (e.g., with respect to the transfer of data and program instruction sets to the smartphone 200).

With reference again to FIG. 3A, in a representative embodiment the second set of patient monitoring devices 110b is provided or carried by, and operates in association with or as part of, a wristband-type device having a processing unit; a memory storing data and program instructions executable by the processing unit, including a sleep monitoring app configured for monitoring patient sleep-related variables; a set of input/output elements for receiving patient input and providing visual or graphical output; and a data communication interface by which the wristband-type device can communicate with the smartphone 200. Such a wristband-type device and the second set of patient monitoring devices 110b provided or carried thereby can be similar or analogous to, be based on, or be a conventional wrist-worn device, as described above.

In this representative embodiment, the set of mental/behavioral health variable monitoring modules 330, when executed/executing, is configured for monitoring the values of particular sleep variables, mobility variables, and sociability variables for the patient. Analogously, the set of mental/behavioral health anomaly detection modules 340, when executed/executing, is configured for detecting patient behavioral and/or mental health anomalies based upon or using data derived from the monitoring of the patient's sleep variable values, mobility variable values, and sociability variable values. Such anomalies can be individual variable anomalies, or joint/multi-variable anomalies. In various embodiments, the set of mental/behavioral health anomaly detection modules 340 can include machine learning modules providing program instruction sets which when executed perform machine learning processes or operations in accordance with particular machine learning models, such as described in further detail below.

Representative aspects of sleep, mobility, and sociability variable monitoring, the detection of particular patient behavioral and/or mental health anomalies correlated therewith, and non-limiting representative manners of responding to detected anomalies are described in detail hereafter.

Aspects of Patient Sleep Monitoring Processes

In various embodiments, sleep anomaly detection analysis can be based on patient-specific analysis (e.g., during a current 7-day observation period). Specifically for sleep, it may involve the analysis of aggregate statistics such as sleep duration and sleep quality of the individual patient, taking into account what is learned to be typical for this particular patient, based on 30-day prior averaging, and what are the normal sleep related variabilities for the individual patient. These aggregate sleep variables are illustrations and are described without limitation, as individuals having ordinary skill in the relevant art will recognize that additional/other aggregate sleep variables such as average time of going to bed could also be used.

The patient's sleep duration sample mean is taken during the current 7-day observation period (SD₇x̅), and the sample standard deviation of patient's sleep duration is taken during the 30-day prior (SD₃₀σ). These determine baseline readings and allow assessment of deviation from baseline of the current patient's observed sleep during the current 7-day observation period. The 30-day averaging baseline can be taken as the values derived from 38 days prior until 8 days prior, as this constitutes a typical or baseline period for a patient.

Aspects of Sleep Duration Anomaly Detection

In some embodiments, a Sleep Duration Anomaly (SD_{A}) can be defined as Sleep Duration during the current 7-day observation period (SD₇x̅) that is greater than the 30-day prior mean (SD₃₀x̅)+2 standard deviation of the 30-day prior (2SD₃₀σ) or less than the 30-day prior mean (SD₃₀x̅)−2 standard deviation of the 30-day prior (2SD₃₀σ):
SD_{A} is present when SD₇x̅>SD₃₀x̅+2SD₃₀σ or SD₇x̅<SD₃₀x̅−2SD₃₀σ

Aspects of Sleep Quality Anomaly Detection

There are different ways of defining sleep quality in the literature. Sleep quality is defined in accordance with particular embodiments of the present disclosure as a measure of sleep efficiency (measured in percentage) defined by the ratio of total-sleep-time to time-in-bed. Good sleep quality is defined as sleep with efficiency (η) of 85% or greater, although values other than 85% may also be used, for example 70% or 80%.

A similar anomaly detection calculation (as that for sleep duration, described above) is performed for sleep quality during the current 7-day observation period by comparison with the 30-day prior sleep quality baseline.

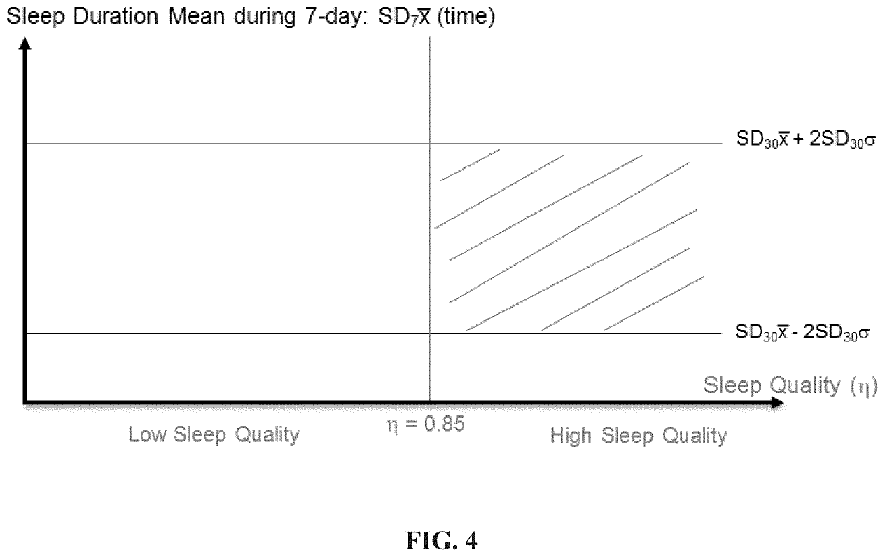

FIG. 4 is a graph corresponding to a manner of determining whether sleep quality anomaly exists based on the aforementioned variables in accordance with an embodiment of the present disclosure. In FIG. 4, the shaded region is considered as a “safe” baseline region, in which the sleep duration mean during the current 7-day observation period is within 2standard deviations (2SD₃₀σ) of the 30-day prior sleep duration mean (SD₃₀x̅) with high quality sleep (i.e., sleep efficiency equal to or greater than 85%). The area outside the shaded region can be considered as anomalous regions.

Aspects of Patient Mobility Monitoring Processes

Mobility in the context of several embodiments of the present disclosure includes aggregate mobility variables such as “time away from home” and “radius of gyration travelled from home.” These aggregate variables are illustrations and are described without limitation. Additional/other aggregate mobility variables such as average time of going out or returning home could also be used, in a manner that individuals having ordinary skill in the relevant art will readily comprehend.

This first requires a definition of “home”: home is defined as the average of the location at which the patient was found most often at the times of 02:00 and 06:00 hours according to their local time zone, during weekdays. These location observations are derived from location information which could be obtained from geolocation signals (e.g., Global Positioning Satellite GPS)) or Network Equipment (e.g., cellular and/or Wi-Fi) signals.

Time away from home (TAH) is then defined as the total time periods observed during a sample period of 7 days when the patient is more than a threshold distance (e.g., 300 meters) from their home location. The radius of gyration is defined as the maximum distance travelled from the centroid of the home location observed during the observation period. Because of the wide dynamic variation in radius of gyration, the logarithm (base 10) of distance travelled from home is recorded and noted as log radius of gyration (LRG).

The associated mobility anomaly detection analysis is then based on an observation of sample mean of the current 7-day observation period, specifically time away from home and radius of gyration travelled from home. This is compared with what has been learned to be typical for this particular patient based on the prior 30-day period during which a sample mean and sample variability of the individual patient is recorded for these mobility features.

Aspects of Mobility Anomaly Detection

A Time Away from Home Anomaly (TAH_{A}) can be determined to be present when the current 7-day observation period (TAH₇x̅) has a sample mean of less than 30 minutes, or is more than 2 sample standard deviations (2TAH₃₀σ) above the 30-day prior sample mean value (TAH₃₀x̅). The lower limit of 30 minutes is selected as an arbitrary cut-off value to account for patient who leaves home for a short amount of time (and thus less likely to have meaningful social interaction or is home-bound) taking into account some natural variations or noise in locational resolution accuracy. Hence:
TAH_{A} is present when TAH₇x̅>TAH₃₀x̅+2TAH₃₀σ, or TAH₇x̅<30 minutes.

Radius of Gyration during the current 7-day observation period, i.e., LRG₇, is compared with LRG₃₀x̅.

Similarly for Radius of Gyration, the Anomaly LRG_{A} is determined to be present when the current 7-day observation period LRG₇ is found to be greater than the 30-day prior mean x̅LRG₃₀+2 standard deviation of the 30-day prior (2σLRG₃₀) or less than the 30-day prior mean (x̅LRG₃₀)−2 standard deviation of the 30-day prior (2σLRG₃₀). That is,
LRG_{A} is present when LRG₇>x̅LRG₃₀+2σLRG₃₀ or LRG₇<x̅LRG₃₀−2σLRG₃₀

Note that the lower limit of 30 minutes is described without limitation and is configured to allow for some errors in geolocation, which could occur. Note also that the algorithm described here is illustrative, and similar algorithms accomplishing a similar, an analogous, or the same purpose will be readily apparent to those with ordinary skill in the relevant art.

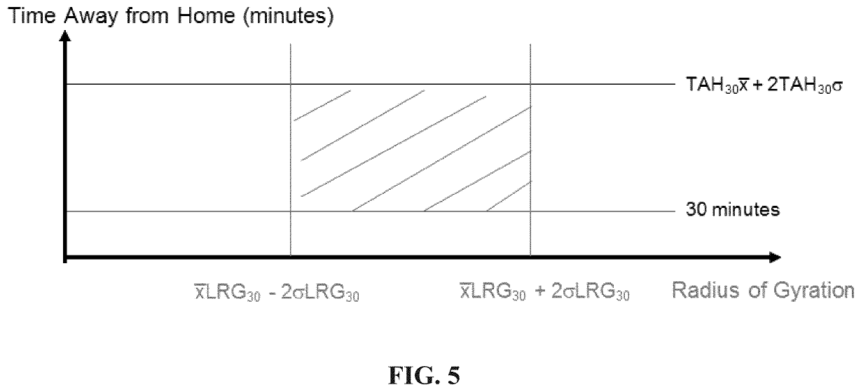

FIG. 5 is a graph corresponding to a manner of determining whether the representative or exemplary mobility anomaly exists based on the aforementioned variables in accordance with an embodiment of the present disclosure. In FIG. 5, the shaded region is considered as the safe baseline region, in which the time away from home during the current 7-day observation period is greater than 30 minutes and smaller than 2 standard deviation (2TAH₃₀σ) of the 30-day prior time away from home mean (TAH₃₀x̅) with the radius of gyration within the 2 standard deviation (2σLRG₃₀) of the 30-day prior mean (x̅LRG₃₀). The area outside the shaded region can be considered as an anomalous region.

Aspects of Patient Sociability Monitoring

Sociability monitoring in various embodiments includes call (audio/video) and texting/messaging in-degree and out-degree and call (audio/video) and texting/messaging reciprocity. These variables provide a summary on a periodic basis of the number of distinct communication partners from whom the patient is contacted (in-degree) (via text/message and audio/video call) and the number of distinct communication partner to whom the patient contacts (out-degree).

Reciprocity has been defined as a measure reflecting the balance between incoming and outgoing communication flows in the literature. Herein, inbound reciprocity can be defined as a percentage of incoming calls (audio/video) that are answered or returned within an hour by the subject patient, and percentage of texts/messages that are responded to within an hour. Similarly, outbound reciprocity can be defined as the portion of time that outgoing calls or messages are responded to be a correspondent of the subject patient. The content of the actual message or identity or contact information of the counterparty is not recorded or transmitted, thus preserving privacy.

The associated sociability anomaly detection analysis (during the current 7-day observation period) is based on patient-specific analysis and specifically for sociability: call and texting/messaging in-degree, out-degree and reciprocity, taking into account what is learned to be typical for this particular patient (based on the 30-day prior observation period as a baseline), i.e., what are the sample means and variabilities of the individual patient for these sociability features.

Aspects of Sociability Anomaly Detection

In-Degree Anomaly (I_{A}) can be defined as Call/Texting/Messaging In-Degree during the current 7-day observation period (I₇x̅) that is greater than the 30-day prior mean (I₃₀x̅)+2 standard deviation of the 30-day prior (2I₃₀σ) or less than the 30-day prior mean (I₃₀x̅)−2 standard deviation of the 30-day prior (2I₃₀σ).
I_{A} is present when I₇x̅>I₃₀x̅+2I₃₀σ or I₇x̅<I₃₀x̅−2I₃₀σ

Out-Degree Anomaly (O_{A}) can be defined as Call/Texting/Messaging Out-Degree during the current 7-day observation period (O₇x̅) that is greater than the 30-day prior mean (O₃₀x̅)+2 standard deviation of the 30-day prior (2O₃₀σ) or less than the 30-day prior mean (O₃₀x̅)−2 standard deviation of the 30-day prior (2O₃₀σ).
O_{A} is present when O₇x̅>O₃₀x̅+2O₃₀σ or O₇x̅<O₃₀x̅−2O₃₀σ

Inbound Reciprocity Anomaly (IR_{A}) can be defined as Call/Texting/Messaging Reciprocity during the current 7-day observation period (IR₇x̅) that is greater than the 30-day prior mean (IR₃₀x̅)+2 standard deviation of the 30-day prior (2IR₃₀σ) or less than the 30-day prior mean (IR₃₀x̅)−2 standard deviation of the 30-day prior (2IR₃₀σ).
IR_{A} is present when IR₇x̅>IR₃₀x̅+2R₃₀σ or IR₇x̅<IR₃₀x̅−2IR₃₀σ, and this gives the corresponding definition for OR_{A}.

Sociability Anomaly is defined as the presence of at least one of the following: In-Degree Anomaly (I_{A}), or Out-Degree Anomaly (O_{A}), or Inbound Reciprocity Anomaly (IR_{A}), or Outbound Reciprocity Anomaly (OR_{A}). While the description herein provides a simple way of combining these indicators to obtain a Sociability Anomaly score, it will be apparent to an individual having ordinary skill in the relevant art that there are a wide variety of logistic regression and machine learning techniques that can additionally or alternatively be used to obtain an overall score.

Note that the admissible range of 2standard deviations is described without limitation, and is configured to allow for natural deviations in sociability, which could occur. Note also that the algorithm described here is illustrative, and similar algorithms accomplishing a similar, an analogous, or the same purpose will be readily apparent to those with ordinary skill in the relevant art.

Aspects of Overall Patient Anomaly Detection Modules and Processes

It is understood that regardless of the choice of allowed variation, or anomaly, some false alarms in the above-described individual univariate anomaly detections can and will occur. These false alarms are generally not problematic as they will result in harmless suggestions. However if these false alarms and suggestions are excessively prevalent they could result in annoyance, or so-called “alert fatigue”. This potential shortcoming can be dealt with in one or several ways in accordance with particular embodiments of the present disclosure. A first way is by adjusting the parameters or nature of the univariate alert algorithms as illustrated and described by the alternatives listed above. The second way is by the combining these alerts into a combined or consensus algorithm, as described immediately below. The third way is using machine learning to automatically tune these parameters to an individual which is further described below in the present disclosure.

An overall anomaly detection process in accordance with several embodiments of the present disclosure takes into account multi-variable anomalies, which in the context of the representative examples considered herein include or correspond to the combination of the sleep anomaly, mobility anomaly and sociability anomaly in a way that brings out the potential of each variable but also seeks a consensus or threshold of these indicators.

A simple overall anomaly detection algorithm threshold is provided for purpose of illustration:
for a seriously ill patient: one or more of the anomalies detected results in an alarm condition; and
for a moderately ill patient: two or more of the anomalies detected results in an alarm condition.

There are also other or more advanced processes or methods that can learn more complex patterns which could be either alarm-generating or non-alarm-generating. An example of a non-alarming double anomaly in single variables would be a large locational gyration combined with sleep disruption: this can be occasioned by intercontinental travel that results in jet lag, with no resulting alarm. On the other hand, a patient that stays at home and also stops communicating would result in an alarm. Hence, for purpose of simplicity and aiding clarity, the description herein describes the use of a general learning algorithm that can learn complex patterns, and a simple process or method using pre-computed features and a neural network which can accommodate the jet lag example given above.

In view of the description herein, embodiments in accordance with the present disclosure can provide personalized and contextualized processes that adapt to the baseline(s) of an individual patient, and via anomaly detection allow an overall anomaly to be detected without excessive false alarms.

Aspects of Deep-Learning-Based Time-Series Anomaly Detection

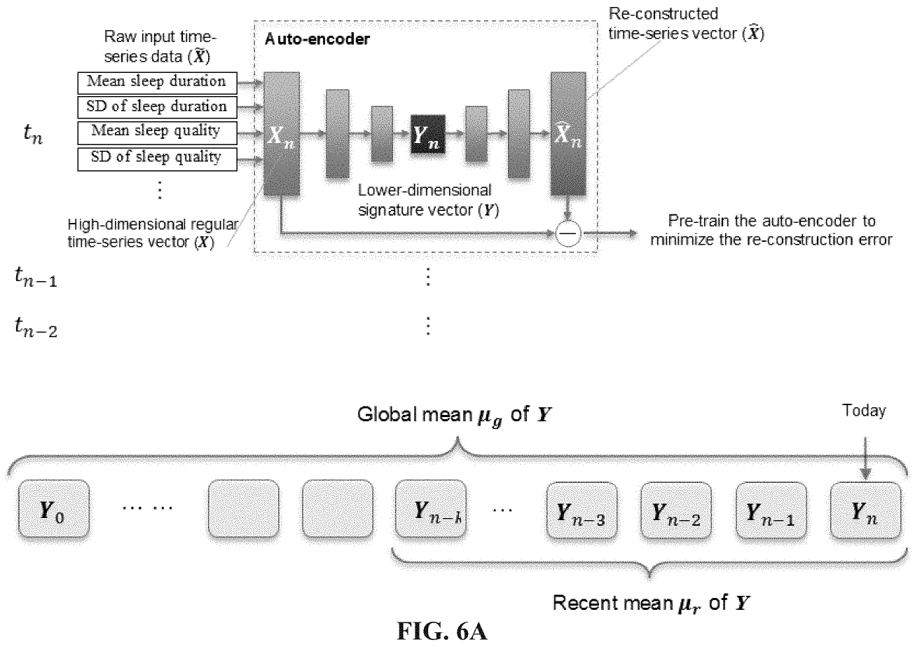

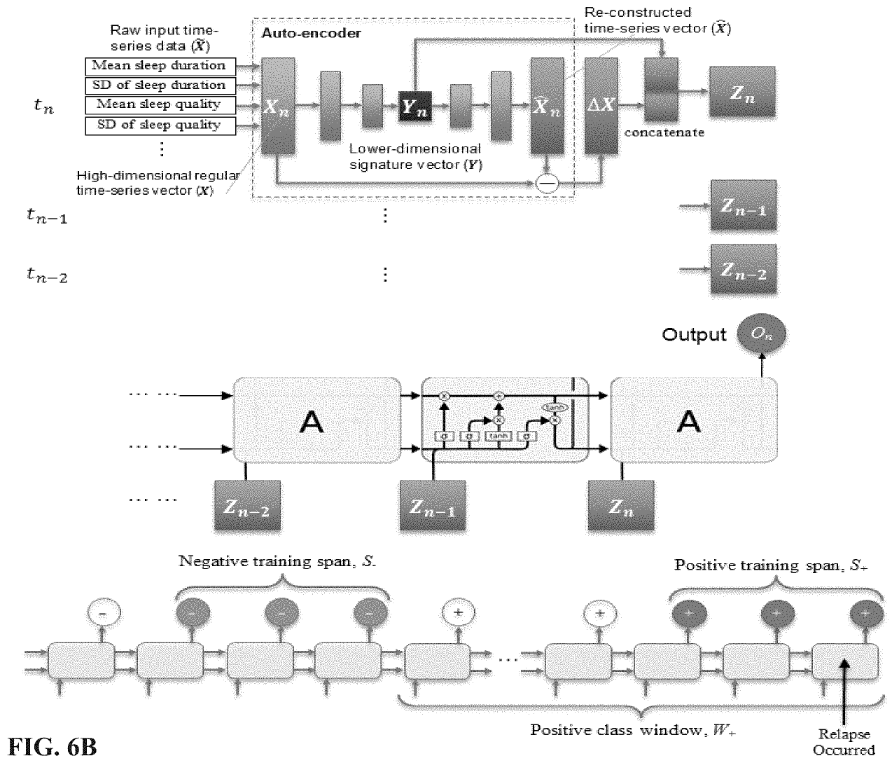

FIG. 6A shows aspects of a first or simple deep learning based overall anomaly detection system in accordance with an embodiment of the present disclosure. FIG. 6B shows aspects of a second or more complex deep learning based overall anomaly detection system in accordance with an embodiment of the present disclosure.

Over every date-time interval (e.g., every day), raw input time-series data (\tilde{X}) which may include every event generated by/captured from the patient (such as time of going to bed, time of going out, time of making a phone call or sending a message) or aggregate variables (such as sleep duration, sleep quality, mobility and sociability features, etc . . . which may be first normalized (subtract by mean and divided by standard deviation) and then concatenated into a high-dimensional regular time-series vector representation (X). Then, unsupervised learning is used to train an auto-encoder (AE⁷) that can reconstruct the high-dimensional vector sequence by first reducing them to a much lower-dimensional time-series vector (Y, called signature representation), i.e., X will first go through a few feed-forward or dense neural network layers with decreasing layer size, to produce the lower-dimensional signature vector (Y) and then go through another few feed-forward or neural network dense layers with increasing layer size to produce the re-constructed vector, \hat{X}, which has the same dimension as X.

Next, the re-construction error vector ΔX is compressed into a shorter vector by way of a dense layer, after which it is concatenated with the signature representation Y to form an input state vector Z. The sequence of input state vector Zₙ, is fed into long short-term memory (LSTM) network to produce an output anomaly value. A threshold is tuned on this value to detect anomalies.

Aspects of Training the Deep Learning System
1. Pre-train the auto-encoder on all data frames from all patients, using the training objective that is to minimize the re-construction error of the high-dimensional time-series vectors from the signature representation. This training takes place by way of gradient descent, also known as back-propagation, which is a technique well known to those who have ordinary skill in the relevant art.
2. Run the auto-encoder on all data frames from all patients to generate Y from all patients.
3. As shown in FIG. 6A, for every patient, compute the global (e.g., all days since the beginning) mean vector (μ_{g}) and covariance matrix (Σ) of Y; and compute the recent (e.g., last 7 days) mean vector (μᵣ) of Y. Tune the anomaly threshold (dₜₕ, the distance away from μ in terms of Mahalanobis distance) to optimize detection accuracy, in a manner understood by individuals having ordinary skill in the relevant art. For operation, compute the μᵣ of Y for the last week, if it is more than dₜₕ away from μ_{g} in Mahalanobis distance (e.g., (μᵣ−μ_{g})^{T}Σ(μᵣ−μ_{g})>dₜₕ), that signals an anomaly.
4. As shown in FIG. 6B, for more advanced and accurate prediction that can capture periodicity (e.g., weekly or annual) and temporal pattern, we can train an LSTM on Y. This approach will require more training data. On any day (e.g., on day n) that a relapse has occurred, we define positive training span (S₊) so that each of the last S₊ days will be used as the positive training data points. We also define positive class window (W₊) and negative training span (S₋) so that each of the first S₋ days in the last (S₋+W₊) days will be used as the negative training data points. For each training data point, the entire vector sequence up to that day since the beginning can be used to train the LSTM. All the span sizes (S₊, S₋, and W₊) need to be tuned to balance the label bias and ensure label reliability. Furthermore, if possible, using continuous values for the output Oₙ can improve performance. They can be obtained, for example, by performing clinical assessment on the patient so as to obtain a clinical rating scale.

Theoretically, training the auto-encoder allows the signature representation Y to be able to extract high-level features from the raw time-series data. These will convey information mainly for seen or directly recognizable (e.g., not hidden) anomalies. Since the AE is trained to compress efficiently and re-construct accurately, unfamiliar patterns tend to result in larger re-construction error(s). Thus, the re-construction error vector ΔX will convey information mainly for unseen anomalies. The LSTM is used to capture temporal patterns that can be indicative of or signal anomalies.

Aspects of Personal Behavioral/Mental Health Management Processes

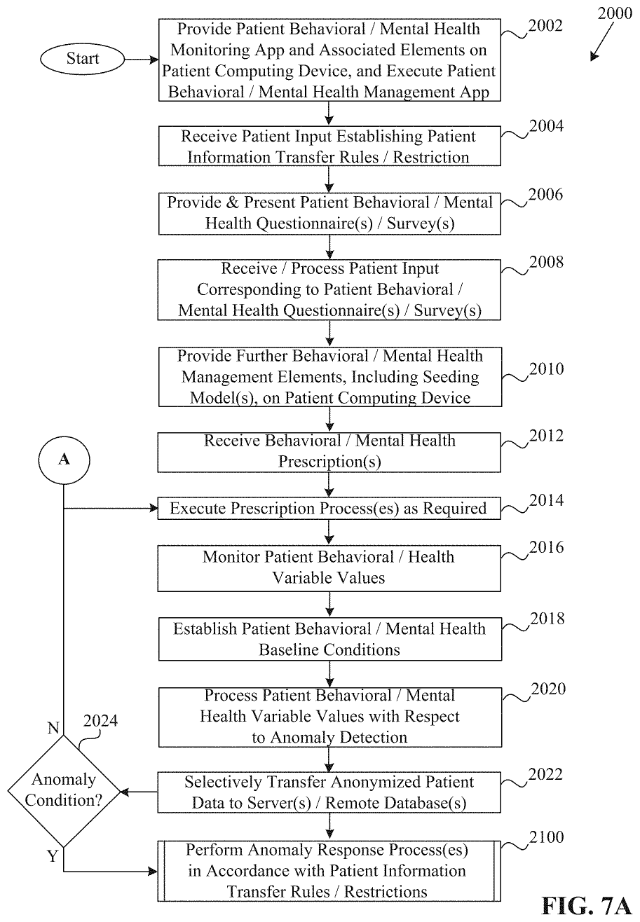

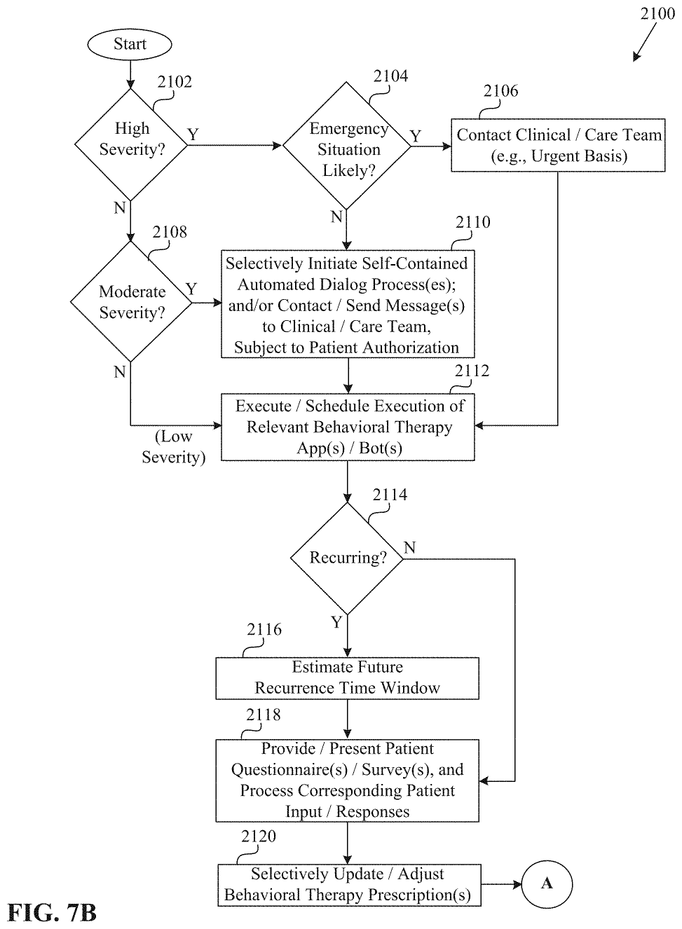

FIGS. 7A-7B are flow diagrams showing aspects of a non-limiting representative process 2000 for personal behavioral/mental health management in accordance with multiple embodiments of the present disclosure, for instance, as managed, directed, and/or performed by the patient behavioral/mental health management app 320 and particular elements associated therewith. Any given embodiment need not include all process portions indicated in FIGS. 7A-7B, and some embodiments can include fewer, additional, and/or other process portions depending upon embodiment details, as individuals having ordinary skill in the relevant art will readily understand.

For purpose of simplicity and to aid understanding, in the following description a patient computing device 200 is defined to include or be a smartphone 200 such as set forth above with respect to FIGS. 3A-3B. Individuals having ordinary skill in the art will also understand that the process 2000 can involve or apply to other or additional patient computing devices 200, depending upon embodiment details.

With additional reference to FIG. 3B, in an embodiment the process 2000 includes a first process portion 2002 involving providing a patient behavioral/mental health management app 320 and an initial set of associated elements on the patient's smartphone 200, and executing the patient behavioral/mental health management app 200. The initial set of associated elements can include, for instance, the data communication manager 380; and possibly one or more social media apps 310 if such are not yet installed on the smartphone 200. The patient behavioral/mental health management app 320 and the initial set of associated elements can be communicated or transferred to the smartphone 200 from a server 1100 or NAS unit 1300 associated therewith by way of the Internet and/or one or more other computer networks 50, for instance, in response to the patient accessing a behavioral/mental health management website (which need not or does not require or retain patient registration/log-in/account details, e.g., in the absence of the patient's explicit permission). A second process portion 2004 involves receiving patient input that establishes patient information/data transfer rules or restrictions, or analogously, a set of patient data communication permissions, that the patient behavioral/mental health management app 320 and data communication manager 380 enforce with respect to data communication or transfer to destinations, data communication networks, systems, and devices external to or remote from the smartphone 200 (e.g., a set of patient information transfer restrictions/permissions that indicate or limit/restrict the types of patient behavioral and/or mental health state related information locally accessible to or locally resident on the patient computing device 200 that the can be transferred to destinations external to the patient computing device 200 or the set of patient-side personal behavioral/mental health management resources 100).

A third process portion 2006 involves providing/presenting one or more behavioral/mental health questionnaires and/or surveys to the patient, where the questionnaire(s) and/or survey(s) can be associated, linked, or included with the patient behavioral/mental health management app 320 or further transferred to the smartphone 200 thereby; and a fourth process portion 2008 involves receiving and processing patient input/responses corresponding to such questionnaires and/or surveys. The third and fourth process portions 2006, 2008 can occur by way of patient interaction with the set of input/output devices 230 and the execution of program instructions by the processing unit 210, in a manner readily understood by individuals having ordinary skill in the relevant art. The patient's input/responses to the questionnaire(s)/survey(s) can establish an initial, most-recent, or current patient behavioral/mental health profile, which can include data associated with or identifying one or more behavioral/mental health conditions and/or symptoms that the patient has experienced or is experiencing on an acute or chronic basis.

A fifth process portion 2010 involves providing an additional set of patient behavioral/mental health management elements on the patient's smartphone 200, possibly or typically based upon the patient's responses to the questionnaire(s)/survey(s). The additional set of patient behavioral/mental health management elements can include, for instance, the patient behavioral/mental health variable monitoring module(s) 330; the patient behavioral/mental health anomaly detection module(s) 340; an initial set of behavioral therapy bots (e.g., CBT chatbots) 350; and initial local behavioral/mental health management database contents. In several embodiments, the patient behavioral/mental health anomaly detection module(s) include program instruction sets configured for implementing one or more types of deep learning models when executed.

A sixth process portion 2012 involves receiving or retrieving one or more current prescriptions for the patient, such as by way of data communication with a particular server 1100 or NAS unit 1300 associated therewith, and/or data communication with a clinical/care team computer system 1500. A prescription in accordance with several embodiments of the present disclosure can include an executable set of program instructions and/or a script that establishes a sequence of automated behavioral therapy activities and/or electronic lessons in which the patient is to engage, and corresponding schedules for such. A current prescription may additionally or alternatively include data and possibly images or videos corresponding to an exercise, dietary, and/or medication regimen/protocol that the patient is to follow, as well as corresponding schedules. A seventh process portion 2014 involves executing a set of current recommended/required automated prescription processes, such as the execution of a particular CBT chatbot 350 in accordance with a given prescription and the schedule corresponding thereto.

An eighth process portion 2016 involves monitoring particular patient behavioral/health variable data or values by the patient behavioral/mental health variable monitoring module(s) 330. In various embodiments, the seventh process portion 2014 includes or is directed to monitoring the patient's mobility, sleep, and sociability variables, for instance, in a manner described above.

A ninth process portion 2018 involves establishing patient behavioral/mental health baseline conditions or values corresponding to the monitored variables, for instance, in a manner set forth above for the patient's mobility, sleep, and sociability variables. Such baseline conditions can correspond to a most-recent time period or interval, such as a particular span of hours, days, or weeks, as will be understood by individuals having ordinary skill in the relevant art.

A tenth process portion 2020 involves processing the patient's behavioral/mental health variable data or data values with respect to detecting one or more anomalies in the patient's behavioral/mental health, for instance, relative to the aforementioned baseline conditions or values. In association with the tenth process portion 2020, the Patient Behavioral/Mental Health Anomaly Detection Module(s) can perform operations, including machine learning operations, such as described above.

An eleventh process portion 2022 involves selectively transferring anonymized patient data corresponding to monitored variable values to a set of servers 1100 and/or remote databases, in the event that such data transfer is permitted in accordance with the patient data transfer rules/restrictions.

A twelfth process portion 2024 involves determining whether an anomaly condition exists (e.g., one or more anomalies in the patient's behavioral/mental health have been detected), based on the tenth process portion 2020. If not, the process 2000 can return to the seventh process portion 2014; otherwise, the process 2000 can transition to a twelfth process portion 2100 to address the anomaly condition.

More particularly, FIG. 7B is a flow diagram illustrating aspects of an anomaly response process 2100, corresponding to the twelfth process portion 2100 of FIG. 7A, in accordance with an embodiment of the present disclosure. In multiple embodiments, the anomaly response process 2100 involves a first process portion 2102 that determines whether the anomaly condition under consideration is a high severity anomaly condition. In some embodiments, a high severity anomaly can be defined as an anomaly that involves more than two variables being monitored, i.e., three or more variables. A high severity anomaly can correspond to or be indicated by separate anomalies in three variables, i.e., three variables considered and processed separately; or three variables considered and processed jointly (e.g., as inputs to a deep learning model).

If a high severity anomaly condition exists, a second process portion 2104 involves determining whether the high severity anomaly condition corresponds to an emergency situation or likely emergency situation. This determination can involve or be based on the patient's most recent/current behavioral/mental health profile. For instance, if the patient has a history of significant (e.g., life threatening) self-harm behaviors or suicidal ideation, an emergency situation may be likely or highly likely. However, if the patient does not have such a history, but rather has a history of particular obsessive/compulsive disorder behaviors, then an emergency situation is not likely.

If an emergency situation is likely, a third process portion 2016 involves contacting the patient's clinical/care team, such as by way of one or more telephone calls and/or emergency alert messages.

In the event that an emergency situation is not likely, the second process portion 2104 can transition to a fifth process portion 2110, as further detailed below.

If in association with the first process portion 2102 a high severity anomaly condition does not exist, a fourth process portion 2108 involves determining whether a moderate severity anomaly condition exists. In embodiments, a moderate severity anomaly can be defined as an anomaly that involves only two variables being monitored, i.e., fewer than three variables. A moderate severity anomaly can correspond to or be indicated by separate anomalies in two variables, i.e., two variables considered and processed separately; or two variables considered and processed jointly (e.g., as inputs to a deep learning model).

If in association with the fourth process portion 2108 a moderate severity anomaly condition exists, or after the second process portion 2104 in the event that an emergency situation does not exist in the context of a high severity anomaly condition, the aforementioned fifth process portion 2110 can involve selectively (a) initiating a completely self-contained automated dialog process (e.g., occurring only on the patient's smartphone 200, without the transfer of patient-related data external to the smartphone 200), during which the patient behavioral/mental health management app 320 (i) presents a set of queries to the patient (e.g., “Are you OK?”, or “Are you currently traveling?” or “Are you currently getting extra rest at home because of a minor illness?”), and (ii) processes the patient's response(s) thereto, which can further clarify, categorize, or define the nature/type and severity of the anomaly condition under consideration; and/or (b) contacting and/or sending messages to the patient's clinical/care team (e.g., by way of data network communication with a clinical/care team computer system 1500), subject to patient authorization. Such patient authorization can be in accordance with the patient data communication rules/restrictions, and can further be explicitly confirmed or limited by way of presenting a set of clinical/care team contact authorization questions on the patient's smartphone 200 (e.g., to which the patient must respond in the affirmative in order for the transfer of one or more types of messages to the patient's clinical/care team to occur). In response to and/or in accordance with patient authorization, the fifth process portion 2110 can involve sending a message such as “I had an episode, but I'm OK” to the clinical/care team. Such a message can indicate that a non-trivial anomaly condition has been detected, yet the patient has not abandoned use of or remains engaged with their behavioral/mental health management app 320.

If in association with the fourth process portion 2108 a moderate severity anomaly condition did not exist, indicating a low severity anomaly condition exists, or after the fifth process portion 2110, a sixth process portion 2112 can involve executing and/or scheduling the execution of one or more behavioral therapy apps/bots (e.g., CBT chatbots) 350 that can aid the patient in addressing, managing, and/or overcoming the stressors and/or situational trigger(s) that led to the anomaly condition under consideration.

A seventh process portion 2114 can involve determining whether the anomaly condition under consideration is or is expected to be a recurring or periodic anomaly condition, which can be associated with a particular type of patient behavioral/mental condition (e.g., a drug addiction relapse), or other factors such as seasonality (e.g., which can affect the severity of depression-related disorders). Such a determination can involve or be based on a deep learning model, for instance, as described above with respect to FIG. 6B. If the anomaly condition under consideration is or is expected to be a recurring or periodic anomaly condition, an eighth process portion 2116 can involve estimating a next expected/likely recurrence or relapse time period or window.

A ninth process portion 2118 can involve providing/presenting one or more behavioral/mental health questionnaires and/or surveys to the patient, and processing the patient's inputs/responses thereto. Based on the patient's current/most-recent anomaly condition and/or historical anomaly conditions, some or each the questionnaire(s)/survey(s) can be the same as or different from the questionnaire(s) and/or survey(s) previously presented to the patient.

A tenth process portion 2120 can involve updating or adjusting one or more patient prescriptions based on the patient's current/most-recent anomaly condition and/or historical anomaly conditions and/or processing the patient's questionnaire/survey inputs/responses. For instance, the tenth process portion 2120 can involve identifying or selecting additional/other behavioral therapy apps/bots (e.g., CBT chatbots) 350 that the patient can regularly use for helping the patient to address particular behavioral/mental health issues associated with their most-recent and/or historical anomaly conditions.

Following the tenth process portion 2120, the process 2100 can transfer to the seventh process portion 2014 of FIG. 7A.

The above description details aspects of multiple systems, apparatuses, devices, techniques, processes, and/or procedures in accordance with particular non-limiting representative embodiments of the present disclosure. It will be readily understood by a person having ordinary skill in the relevant art that modifications can be made to one or more aspects or portions of these and related embodiments without departing from the scope of the present disclosure, which is limited only by the following claims.

1. A computerized method for non-revealing monitoring and processing of behavioral and/or mental state related information corresponding to a patient, the method comprising:
(1) providing a patient computing device controlled by the patient, the patient computing device comprising a processing unit comprising integrated circuitry and coupled to each of a memory, a set of input/output devices configured for providing a user interface, and a data communication unit, wherein the memory stores program instruction sets executable by the processing unit including program instruction sets corresponding to each of: (a) a patient behavioral and/or mental health management application program executable by the processing unit, which when executed provides visual or graphical interfaces by which the patient can interact with the patient computing device for self-management of their behavioral and mental health, and selectively communicate with devices and systems external to the patient computing device; (b) a set of patient behavioral/mental health variable monitoring modules, which when executed perform numerical/statistical operations upon patient behavioral, physiological, and/or mental health data; (c) a set of patient behavioral and/or mental health anomaly detection modules, which when executed perform anomaly detection operations in accordance with one or more machine learning or artificial intelligence models by which patient behavioral and/or mental health anomaly conditions can be automatically detected or recognized; (d) a behavioral therapy automaton; and (e) a data communication manager configured for managing or controlling data communication between the patient computing device and external systems, external devices, and data communication networks;
(2) providing a set of patient behavioral and/or physiological parameter monitoring elements, each of which is configured for generating data correlated with the patient's behavioral and/or physiological state at a particular time or during a particular time period, wherein the set of patient behavioral and/or physiological parameter monitoring elements comprises one or more electronic or computing devices configured for data communication with the patient computing device, and/or patient computing device hardware and/or program instruction sets;
(3) initiating execution of the patient behavioral and mental health management application program;
(4) receiving patient input by way of the set of input/output devices and establishing a set of patient data communication permissions that indicates types of patient behavioral and/or mental state information locally resident on the patient computing device that the patient computing device is permitted to communicate to destinations external to the patient computing device;
(5) automatically monitoring patient behavioral and/or physiological parameters over a first time period and generating corresponding first patient behavioral and/or physiological parameter data by way of the set of patient behavioral and/or physiological parameter monitoring elements;
(6) automatically processing the first patient behavioral and/or physiological data to determine a patient behavioral and/or mental health baseline state correlated with the first patient behavioral and/or physiological parameter data;
(7) automatically monitoring patient behavioral and/or physiological parameters over another time period and generating corresponding additional patient behavioral and/or physiological parameter data by way of the set of patient behavioral and/or physiological parameter monitoring elements;
(8) automatically processing the additional patient behavioral and/or physiological parameter data relative to the patient behavioral and/or mental health baseline state and determining whether a patient anomaly condition exists;
(9) in response to the existence of a patient anomaly condition, at least one of automatically:
(a) estimating or determining a severity level corresponding to the anomaly condition;
(b) initiating execution of the behavioral therapy automaton and performing an automated behavioral therapy process based on the patient anomaly condition;
(c) initiating execution of an automated patient dialog process that operates only on the patient computing device, without transfer of patient data and/or patient-identifying data external to the patient computing device; and
(d) selectively initiating data communication with an electronic device or computing device or system corresponding to a care provider or care provider team associated with the patient in accordance with the set of patient data communication permissions.

2. The method of claim 1, further comprising automatically: repeating performing (6) through (9); and updating the patient behavioral and/or mental health baseline state over time based on one or more sets of additional patient behavioral and/or physiological parameter data.

3. The method of claim 2, wherein determining whether a patient anomaly condition exists comprises identifying at least one of: a single variable anomaly corresponding to one patient behavioral and/or physiological parameter, and a multiple variable anomaly corresponding to at least two different patient behavioral and/or physiological parameters.

4. The method of claim 1, wherein the set of patient data communication permissions pre-approves transfer of at least some types of anonymized patient data external to the patient computing device, and wherein the method further comprises: automatically generating anonymized patient behavioral and/or mental health data correlated with at least portions of one or more sets of additional patient behavioral and/or physiological parameter data; and communicating the anonymized patient behavioral and/or mental health data to a remote computer system or remote data store associated with a behavioral and/or mental health care provider or care provider team for the patient in accordance with the set of patient data communication permissions.

5. The method of claim 1, wherein monitoring patient behavioral and/or physiological parameters over the first time period and the second time period comprises monitoring at least some of patient: heart and/or pulse rate; body temperature; breathing rate; body weight; body fat percentage; blood pressure; geolocation; movement or mobility frequency, speed, range, and/or range variability; in-bed or sleep related parameters; circadian rhythms; electroencephalography (EEG) signals; and social media, Internet browser, short message service (SMS) messaging activity, and e-mail usage.

6. The method of claim 5, wherein monitoring patient behavioral and/or physiological parameters over the first time period and the second time period comprises monitoring data corresponding to each of patient: sleep duration, sleep quality, mobility, and sociability.

7. The method of claim 2, further comprising:
presenting the patient with one or more behavioral and/or mental health surveys and/or questionnaires and receiving patient survey and/or questionnaire input corresponding thereto by way of the set of input/output devices;
automatically processing the patient survey and/or questionnaire input and determining a current patient behavioral and/or mental health profile; and
based on the processed patient survey and/or questionnaire input, downloading one or more of the set of patient behavioral/mental health variable monitoring modules, the set of patient behavioral and/or mental health anomaly detection modules, the behavioral therapy automaton, program instruction sets corresponding to a behavioral and/or mental health prescription, and one or more electronic behavioral and/or mental health lessons from a set of remote computing systems associated with a behavioral and/or mental health care provider for the patient.

8. The method of claim 7, wherein the set of patient behavioral and/or mental health anomaly detection modules is seeded to establish what is normal, what is normal variability, and what is anomalous for a group of individuals.

9. The method of claim 7, wherein the behavioral and/or mental health prescription comprises:
a set of program instructions and/or a script executable by the processing unit, which establishes a sequence of automated behavioral therapy activities and/or electronic lessons in which the patient is to engage, and corresponding schedules for the automated behavioral therapy activities and/or electronic lessons; and optionally
data, images, and/or videos corresponding to an exercise, dietary, and/or medication protocol that the patient is to follow.

10. The method of claim 9, further comprising:
determining that a patient anomaly condition is a recurring patient anomaly condition exists, which the patient recurrently experiences over time; and
after determining that a recurring patient anomaly condition exists, at least one of:
(a) automatically estimating a next recurrence time period; and
(b) presenting the patient with one or more additional behavioral and/or mental health surveys and/or questionnaires and receiving additional patient input corresponding thereto by way of the set of input/output devices;
automatically processing the additional patient input; and
automatically adjusting or updating the behavioral and/or mental health prescription based on the processed additional patient input.

11. A system for non-revealing monitoring and processing of behavioral and/or mental state related information corresponding to a patient, the system comprising:
a patient computing device controlled by the patient, the patient computing device comprising a processing unit comprising integrated circuitry and coupled to each of a memory, a set of input/output devices configured for providing a user interface, and a data communication unit, wherein the memory stores behavioral and/or health program instruction sets executable by the processing unit including program instruction sets corresponding to each of: (a) a patient behavioral and/or mental health management application program executable by the processing unit, which when executed provides visual or graphical interfaces by which the patient can interact with the patient computing device for self-management of their behavioral and mental health, and selectively communicate with devices and systems external to the patient computing device; (b) a set of patient behavioral and/or mental health variable monitoring modules, which when executed perform numerical or statistical operations upon patient behavioral, physiological, and/or mental health data; (c) a set of patient behavioral and/or mental health anomaly detection modules, which when executed perform anomaly detection operations in accordance with one or more machine learning or artificial intelligence models by which patient behavioral and/or mental health anomaly conditions can be automatically detected or recognized; (d) a behavioral therapy automaton; and (e) a data communication manager configured for managing or controlling data communication between the patient computing device and external systems, external devices, and data communication networks; and
a set of patient behavioral and/or physiological parameter monitoring elements, each of which is configured for generating data correlated with the patient's behavioral and/or physiological state at a particular time or during a particular time period, wherein the set of patient behavioral and/or physiological parameter monitoring elements comprises one or more electronic or computing devices configured for data communication with the patient computing device, and/or patient computing device hardware and/or program instruction sets,
wherein the patient behavioral and/or mental health program instruction sets, when executed, cause the patient computing device to:
(1) initiate execution of the patient behavioral and mental health management application program;
(2) receive patient input by way of the set of input/output devices and establish a set of patient data communication permissions that indicates types of patient behavioral and/or mental state information locally resident on the patient computing device that the patient computing device is permitted to communicate to destinations external to the patient computing device;
(3) automatically monitor patient behavioral and/or physiological parameters over a first time period and generating corresponding first patient behavioral and/or physiological parameter data by way of communication with the set of patient behavioral and/or physiological parameter monitoring elements;
(4) automatically process the first patient behavioral and/or physiological data to determine a patient behavioral and/or mental health baseline state correlated with the first patient behavioral and/or physiological parameter data;
(5) automatically monitor patient behavioral and/or physiological parameters over another time period and generating corresponding additional patient behavioral and/or physiological parameter data by way of communication with the set of patient behavioral and/or physiological parameter monitoring elements;
(6) automatically process the additional patient behavioral and/or physiological parameter data relative to the patient behavioral and/or mental health baseline state and determining whether a patient anomaly condition exists;
(7) in response to the existence of a patient anomaly condition, at least one of automatically:
(a) estimate or determining a severity level corresponding to the anomaly condition;
(b) initiate execution of the behavioral therapy automaton and performing an automated behavioral therapy process based on the patient anomaly condition;
(c) initiate execution of an automated patient dialog process that operates only on the patient computing device, without transfer of patient data and/or patient-identifying data external to the patient computing device; and
(d) selectively initiate data communication with an electronic device or computing device or system corresponding to a care provider or care provider team associated with the patient in accordance with the set of patient data communication permissions.

12. The system of claim 11, wherein the patient behavioral and/or mental health program instruction sets, when executed, further cause the patient computing device to:
repeatedly perform (5) through (7); and
update the patient behavioral and/or mental health baseline state over time based on one or more sets of additional patient behavioral and/or physiological parameter data.

13. The system of claim 12, wherein the set of patient behavioral and/or health anomaly detection modules is configured for identifying at least one of: a single variable anomaly corresponding to one patient behavioral and/or physiological parameter, and a multiple variable anomaly corresponding to at least two different patient behavioral and/or physiological parameters.

14. The system of claim 11, wherein the data communication module is configured to control communication of patient data and patient-identifying data to destinations external to the patient computing device in accordance with the set of patient data communication permissions.

15. The system of claim 11, wherein the set of patient behavioral and/or physiological parameter monitoring modules is configured for monitoring at least some of patient: heart and/or pulse rate; body temperature; breathing rate; body weight; body fat percentage; blood pressure; geolocation; movement or mobility frequency, speed, range, and/or range variability; in-bed or sleep related motion; circadian rhythms; electroencephalography (EEG) signals; and social media, Internet browser, short message service (SMS) messaging activity, and e-mail usage.

16. The system of claim 15, wherein the set of patient behavioral and/or physiological parameter monitoring modules is configured for monitoring each of patient sleep patterns, patient mobility, and patient social media usage.
